(19) United States
(12) Patent Application Publication
(10) Pub. No.: US 2012/0301889 A1
(43) Pub. Date: Nov. 29, 2012

(54) Method and reagents for identifying pluripotent stem cells

(75) Inventors: Linda E. Iverson, Pasadena, CA (US); Sailesh Gopalakrishna-Pillai, Azusa, CA (US)

(73) Assignee: City of Hope, Duarte, CA (US)

(21) Appl. No.: 13/506,850

(22) Filed: May 21, 2012

Related U.S. Application Data
(60) Provisional application No. 61/457,728, filed on May 20, 2011.

(51) Int. Cl. C12Q 1/68; G01N 33/573; C07K 16/18; G01N 33/566; G01N 33/577

(52) U.S. Cl. 435/6.12; 435/6.18; 435/6.1; 435/7.1; 530/387.9; 530/391.3

(57) ABSTRACT

The present invention relates to methods for distinguishing pluripotent stem cells from partially differentiated, or spontaneously differentiated cells, and to reagents for use in such methods. In particular, the method enables the detection of alternatively spliced transcripts and the polypeptides encoded thereby, which are uniquely associated with, or present at a higher level in pluripotent stem cells than in cells which have partially differentiated. Reagents for use in the method include nucleic acids which bind the alternatively spliced transcript or which amplify the alternatively spliced transcript, and antibodies which bind the polypeptide product of the alternatively spliced transcript.

### CROSS REFERENCE TO RELATED APPLICATIONS

This application is related to and claims priority under 35 U.S.C. §119(e) to U.S. Provisional Application No. 61/457,728, filed May 20, 2011 and incorporated by reference herein in its entirety.

### FIELD OF THE INVENTION

The present invention relates to methods for distinguishing pluripotent stem cells from partially differentiated cells, or spontaneously differentiated cells, and to reagents for use in such methods. In particular, the method enables the detection of alternatively spliced transcripts and the polypeptides encoded thereby, which are uniquely associated with, or present at a higher level in pluripotent stem cells than in cells which have partially differentiated.

### BACKGROUND OF THE INVENTION

Advancements in the studies of human embryonic stem cells (hESCs) and induced pluripotent stem cells (iPSCs) have created new opportunities for basic research and regenerative medicine (Nicholas and Kriegstein, 2010). These cells have wide-ranging applications in cell replacement therapies, development of model systems for studying diseases and drug testing. To realize the full potential of pluripotent stem cells (PSCs), however, many hurdles must be overcome.

For example, pluripotent stem cells (PSCs) propagated in vitro often spontaneously differentiate into unknown or undesired cell types. Although spontaneous differentiation of mouse ES cells can be prevented by supplementing the media with leukemia inhibitory factor (LIF), LIF does not prevent differentiation of human ES cells and comparable factors have not been identified (Odorico et al, 2001). In addition, limitations in the ability to detect dynamic changes in PSCs during self-renewal and early stages of differentiation are due primarily to a dearth of reliably accurate and sensitive detection assays. Additional reagents are needed to detect loss of pluripotency and to refine culture conditions that promote maintenance of the pluripotent state.

Researchers studying PSC's also face additional challenges. For example, although normal hESCs control their rate of proliferation, retain the ability to self-renew, and preserve pluripotency and at the same time maintain genomic integrity, both hESCs and iPSCs have a propensity to acquire karyotypic abnormalities in culture (Blasco et al., 2011; Taapken et al., 2011). Directed differentiation of aneuploid hESCs gives rise to stem-like cells with remarkable similarities to cancer stem cells (Gopalakrishna-Pillai and Iverson, 2010), suggesting mechanisms regulating self-renewal, differentiation and proliferation are shared by normal and cancer stem cells (Clarke and Fuller, 2006). For example, recent reports indicate that the tumor suppressor p53, which plays a crucial role in maintaining genome stability, is also involved in maintenance of stem cell pluripotency and nuclear reprogramming (Deng and Xu, 2009). However, very little else is known about how hESC's and iPSC's control proliferation, self-renew, and maintain genomic integrity, and thus there is a need in the art for developing reliable assays and reagents for identifying these processes.

The transcriptional profiles of hESC and iPSC genes that regulate self-renewal, asymmetric cell division and signaling pathways are currently being characterized; however, relatively little is known about other mechanisms that may be controlling gene expression in these cells, such as post-transcriptional gene regulatory mechanisms. Bioinformatic analysis of expressed sequence tags deposited in public databases indicate that hESCs express alternatively spliced variants of many genes that play important roles in signaling pathways and that have been implicated in development and differentiation (Pritsker et al, 2005).

Hybridization of RNA isolated from hESCs and neural progenitors to exon microarrays identified several genes for which expression ratios of alternative splice variants differed during neural differentiation (Yeo et al, 2007). The widespread alternative splicing observed across various classes of hESC genes, including multiple components of signaling pathways, strongly suggests that alternative splicing is a key regulator of hESC gene expression. Despite these findings, little effort has been directed at investigating alternatively spliced variants as unique markers of pluripotency, specific differentiation stages or cell type lineages.

Additional problems in human iPSC research include the infrequency of iPSC generation and the inefficiency of differentiation of iPSC into desired cell types (Kim et al., 2011). This likely reflects the difficulty of reestablishing the complex transcriptional network of a pluripotent cell in the context of a differentiated cell that has already acquired the appropriate transcriptional program. It is not surprising that differences in iPSC and hESC gene expression, and incomplete reprogramming of iPSCs, are often observed (Barrero and Izpisua Belmonte, 2011). Reestablishing stem cell transcriptional programs in a somatic cell is undoubtedly a multi-step process that requires erasing the epigenetic marks of a differentiated cell, then replacing them with the epigenetic marks of a pluripotent cell.

Though some aspects of these processes undoubtedly are controlled at the level of transcription, alternative splicing has also been validated as a major molecular mechanism regulating gene expression in eukaryotes. Transcriptional control results in a gene being ‘on or off’ or ‘high or low’, while alternative splicing results in more subtle effects by modulating the expression of splice variants encoding protein isoforms with similar yet non-identical properties. Thus alternative splicing plays a major role in generating proteome diversity. Interestingly, bioinformatic analysis of expressed sequence tags deposited in public databases indicate that hESCs express alternatively spliced variants of many genes that play important roles in signaling pathways and that have been implicated in development and differentiation (Pritsker et al, 2005). Moreover, alternative splicing has been shown to regulate the delicate balance between stem cell pluripotency and differentiation (Salomonis et al., 2010). That the same splice variant of the same gene can regulate the switch between self-renewal and differentiation and contribute to efficient reprogramming of somatic cells to iPSCs was recently confirmed in an elegant paper (Gabut et al., 2011) demonstrating that alternative splicing of the mouse transcription factor gene, FOXP1, results in a unique splice variant that i) is expressed exclusively in pluripotent cells, ii) promotes self-renewal by stimulating expressing of ‘pluripotency’ genes, iii) inhibits expression of ‘differentiation’ genes, and iv) facilitates iPSC nuclear reprogramming.

Despite these findings, many bottlenecks in human PSC research remain. Among these are an inability to prevent spontaneous differentiation of the cells in culture and the lack of robust, reliably specific reagents that distinguish PSCs from spontaneously differentiated cells (SDCs). Properties such as the ability to self renew or differentiate into cells of all three lineages are hallmarks of pluripotent stem cells that are controlled by exquisite gene regulatory mechanisms that operate at multiple levels, including transcriptional, post-transcriptional, translational and post-translational. Although more than 90% of human genes are alternatively spliced and alternative splicing is a major source of generating proteome diversity (Orengo and Cooper, 2007), its importance in stem cell research has been underappreciated and, to some extent, unrecognized. This may be explained, in part, by the over reliance on cDNA microarrays, which cannot distinguish among alternatively spliced transcripts (Adewumi et al., 2007).

A major obstacle in human stem cell research is the limited number of reagents capable of distinguishing pluripotent stem cells from partially differentiated or incompletely reprogrammed derivatives. Although hESCs and iPSCs express numerous alternatively spliced transcripts, little attention had been directed at developing splice variant-encoded protein isoforms as reagents for stem cell research or at developing reagents and methods for identifying PSC's and differentiating PSC's from SDC's.

Rather than relying on differences in whole gene transcription to identify new markers of pluripotency, there is a need in the art for identifying alternatively spliced, protein-coding exons that are abundantly and uniquely expressed in PSCs.

### SUMMARY OF THE INVENTION

The present invention provides a method for distinguishing pluripotent stem cells from partially differentiated cells by detecting alternatively spliced transcripts and the polypeptides encoded thereby in the pluripotent stem cells. In particular, those transcripts and their protein products that are either uniquely associated with PSCs, or are present at a detectably higher level in PSCs compared to cells which have partially differentiated from PSCs.

The inventors have found that there are a number of different alternatively spliced transcripts which are present at different levels in PSCs than in SDCs. Moreover, certain alternatively spliced transcripts are uniquely associated with PSCs, and therefore provide superior reagents for distinguishing PSCs from SDCs. One such alternatively spliced transcript is from the DNA methyltransferase gene (DNMT3B), and specifically includes exon 10.

In another aspect, the invention provides methods of distinguishing PSCs from SDCs by amplifying and detecting cDNA created from alternatively spliced mRNAs which are preferentially expressed in PSCs. Such alternatively spliced mRNAs may include a particular exon (“exon-included splice variant”) or may not include a particular exon (“exon-excluded splice variant”).

In yet another aspect, the invention provides reagents which detect alternatively spliced transcripts, such as nucleic acids which can hybridize specifically to those alternatively spliced transcripts, and to primer sets which can amplify such alternatively spliced transcripts when used in accordance with polymerase chain reaction (PCR).

In yet another aspect, the invention provides reagents in the form of reporter gene constructs that may be used to distinguish PSC's from SDC's by making use of the alternative splicing pattern of the alternatively spliced transcripts. The construct incorporates reporter genes that are known to those skilled in the art, and thus may be used to visually identify PSC's, thereby differentiating them from SDC's.

In yet a further aspect of the invention, methods and materials are provided for use in the characterization of various features associated with alternatively spliced transcripts. More prefereably, these materials and methods identification of genes effected by the loss of silencing of alternatively spliced transcripts,

Yet a further aspect of the invention is to provide isolated peptides and polypeptides encoded by the alternatively spliced transcripts, as well as antibodies which bind those peptides and polypeptides.

In a further aspect, the invention provides methods of distinguishing PSCs from SDCs by detecting the peptides and polypeptides encoded by the alternatively spliced transcripts using those antibodies.

The foregoing and other objects, features and advantages of the invention will be apparent from the following more particular description of preferred embodiments of the invention.

### BRIEF DESCRIPTION OF THE DRAWINGS

FIG. 1. Human PSCs exhibit differential expression of alternative splice variants as the cells spontaneously differentiate. RT-PCR analysis using exon-specific primers was performed using cDNA isolated from pluripotent stem cells (PSCs) or cells that had undergone spontaneous differentiation for 14-15 days (SDCs). Gene names are on the left and PCR product size (MW) and exon identity (exon) are indicated on the right. 18sRNA was used as control in RT-PCR reactions.

FIG. 2. Exon 10-included DNMT3B splice variant is expressed at higher levels in pluripotent stem cells. A. Depiction of alternative splicing of DNMT3B exon 10 and location of PCR primers for qRT-PCR reactions. B. Quantitative changes in expression of DNMT3B exon 10, as measured by realtime PCR, in undifferentiated pluripotent stem cells (PSCs) or spontaneously differentiated cells (SDCs; 14-15 days), in the H9 hESC, BG01 hESC and foreskin-1 iPSC lines.

FIG. 3. DNMT3B exon 10 and encoded peptide sequence used for immunization. A. Sequence of DNMT3B exon 10 encoding a 15 amino acid peptide used for generating the SG1 peptide antibody. B. Dot blot analysis demonstrating specificity of SG1 antibody relative to pre-immune sera. Decreasing quantities (in □g) of peptide antigen were adsorbed to the membrane, incubated with either the SG1 antibody or pre-immune sera, incubated with secondary antibody and the blot developed as described in Materials and Methods. Pre-immune sera detected no peptide antigen even at the highest concentration.

FIG. 4. DNMT3B exon 10 encoded peptide antibody, SG1, detects pluripotent stem cells. Dual immunofluorescence assay of undifferentiated pluripotent stem cell lines, H9, HES4 and iPSC stained with OCT4 or SG1 antibodies. Phase contrast image of stem cell colonies (Phase) and same colony stained with Hoechst dye (Hoechst; blue), OCT4 antibody (OCT4; green), and SG1 antibody (SG1; red).

FIG. 5. DNMT3B protein containing the exon 10-encoded peptide is expressed only in pluripotent stem cells. Western blot analysis using DNMT3B exon 10 peptide antibody (SG1), OCT4 and GAPDH (control) antibodies to detect proteins expressed in pluripotent stem (PSCs) and spontaneously differentiated (SDCs; 14-15 days) cells.

FIG. 6. SG1 antibody identifies pluripotent stem cells in mixed populations. Mixed populations of pluripotent and early-stage spontaneously differentiated cells (4-5 days minus zbFGF) obtained from (A) BG01 hESC line or (B) foreskin-1 iPSC line were stained with SG1 antibody and compared to cells stained with α-OCT4 polyclonal and two commercially available α-DNMT3B polyclonal antibodies, one from Cell Signaling (CS) and one from Santa Cruz (H-230). Phase contrast image of stem cell colonies (Phase) and same cells stained with Hoechst dye (Hoecsht; blue), α-OCT4 antibody (OCT4; green) and one of three different α-DNMT3B antibodies (DNMT3B; red) as indicated on the right. The α-DNMT3B antibodies used included the custom peptide antibody, SG1 (top), or one of two commercial antibodies, CS (middle) or H-230 (bottom). Compact colonies of pluripotent stem cells are indicated by large arrows, while dispersed spontaneously differentiated cells are indicated by small arrows in the phase contrast images.

FIG. 7. Time course of expression of DNMT3B exon 10 containing transcripts relative to OCT4 transcripts in spontaneously differentiating cells. RNA extracted from H9 cells induced to differentiate by removal of zbFGF from the media for the indicated number of days (0, 3, 6, 9, 12 or 15) was subjected to qRT-PCR analysis. Relative expression levels of OCT4 transcripts (black bars) in comparison to DNMT3B exon 10 containing transcripts (white bars) are plotted as a function of the number of days of spontaneous differentiation. Duplicate qRT-PCR experiments were performed for each sample; the mean of the two experiments is plotted with SEM indicated by the error bars.

FIG. 8. SG1 antibody is superior to OCT4 and TRA-1-60 antibodies at identifying pluripotent stem cells in mixed populations. Mixed populations of pluripotent and spontaneously differentiated cells (4-5 days minus zbFGF) obtained from the H9 hESC line were analyzed by dual immunofluorescence staining using SG1 rabbit polyclonal antibody and mouse monoclonal antibodies to OCT4 (A) or TRA-1-60 (B). A. Brightfield images of stem cell colonies (Brightfield) and same cells stained with Hoechst dye (Hoecsht; blue), α-OCT4 antibody (OCT4; green) and α-DNMT3B exon 10 encoded peptide (SG1; red) are shown. OCT4 and SG1 staining patterns are overlaid (Merge) and the area outlined by the white box in the Merge panel is shown in the Magnification panel to facilitate visualization of precise staining patterns in individual cells. The large arrow in the Magnification panel indicates a cell exhibiting high level expression of both OCT4 and SG1 (in this case, the cell is undergoing mitosis and the SG1 antibody ‘paints’ the chromatids of the dividing cell), the small arrow identifies a cell exhibiting high OCT4 but low SG1 staining, while the large arrowhead indicates a cell still expressing high levels of OCT4 that is not stained by the SG1 antibody. B. Similar analysis as in A (above) using a monoclonal antibody that detects the stem cell marker TRA-1-60 (green). As opposed to the intracellular proteins (above), the TRA-1-60 antibody detects the expected TRA-1-60 expression on the cell surface. While high-level TRA-1-60 expression is detected on almost all cells (both PSCs and SDCs), SG1 staining is more tightly restricted to PSCs or those cells in very early stages of spontaneous differentiation.

FIG. 9. Sequences of exon-specific primers used for semi-quantitative RT-PCR (FIG. 1) or real time PCR analysis (FIGS. 2 and 7)

FIG. 10. Sequences of PCR amplified products, which were either sequenced directly using exon-specific primers or subcloned into StrataClone vector and sequenced using T3 primers.

FIG. 11. Targeted shRNA knockdown (KD) of DNMT3B exon 10 containing transcripts in hESC's results in mitotic defects. Pluripotent BG01 cells were transfected with lentiviral particles expressing either a non-target control shRNA (control shRNA) or a DNMT3B exon 10 specific shRNA (α-exon 10 shRNA) overnight, transductants were selected for puromycin resistance and transduced cells were stained with Hoechst dye (blue) and α-β-tubulin antibody (red) to visualize chromatids and mitotic spindle fibers, respectively. Representative examples of metaphase and anaphase cells are shown.

FIG. 12. Directed differentiation of hESCs into neural progenitor cells. A. hESCs cultured on MEF feeder layer after 5 days. B. Embryoid bodies (EBs) derived from hESCs after 4 days. Neurospheres (NS) derived from EBs after 21 days. D. Neural progenitor cells derived from NS after an additional 7 days in culture.

FIG. 13. RT-PCR validation of DNMT3B exon 10 exclusion during neural-directed differentiation of PSCs.

FIG. 14. Unsupervised hierarchical clustering of RNAs expressed in H9 hESCs treated with control shRNA (two on left) or DNMT3Be10 shRNA (two on right) at p<0.05. Red indicates relatively high, and blue indicates relatively low expression in the heat map.

FIG. 15. Intron sequences located upstream of DNMT3B exons 10 and 11 3′ splice sites.

FIG. 16. DNMT3B exon 10 5′ splice site and intron 10/11 sequence located down stream of exon 10 5′ ss.

FIG. 17. Western blot of polypyrimidine tract binding protein (PTB) expression in H9 hESCs (lane 1), H9 derived neural progenitors (lane 2), STTG1 astrocytoma cells (lane 3), BG01V hESCs (lane 4), and BG01V derived neural progenitors (lane 5).

FIG. 18. GFP expression in CMV-GFP transfected H9 hESCs in the pluripotent stem cell state (A), following differentiation into embryoid bodies (B), and in differentiated neurospheres (C).

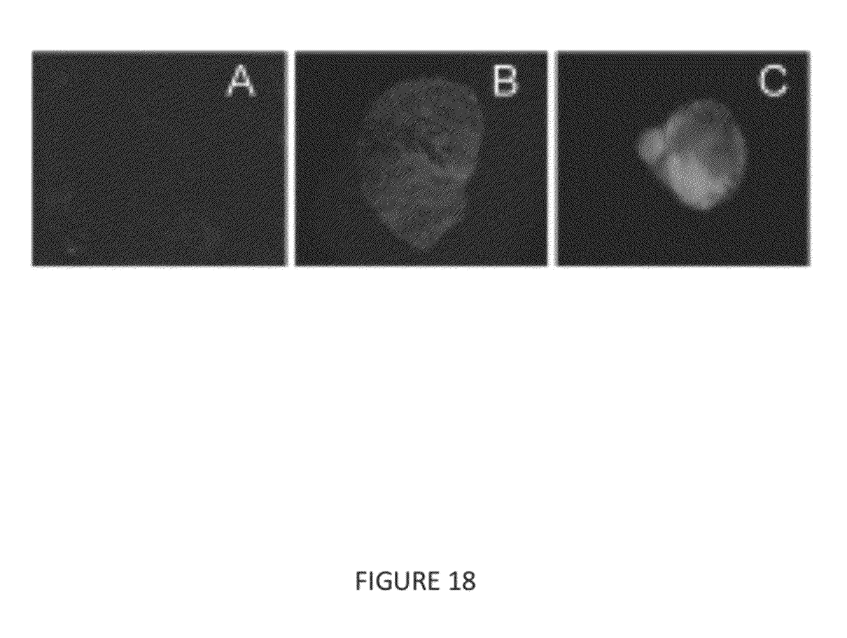

FIG. 19. General design of a DNMT3Be10-GFP splicing reporter construct. CMV promoter is fused to an ATG start codon (with upstream ribosome binding site) followed by a canonical 5′ splice site (GTGAGT). Sequences derived from DNMT3B gene are highlighted in red and include ˜100 nucleotides of intron 9/10 sequences located immediately upstream of the DNMT3B exon 10 3′ ss, DNMT3B exon 10 sequence, ˜100 nucleotides of DNMT3B intron 10/11 downstream of DNMT3B exon 10 5′ ss, an additional ˜100 nucleotides of DNMT3B intron 10/11 upstream of DNMT3B exon 11 3′ ss, and ˜20-40 nucleotides of DNMT3B exon 11. This sequence is then fused to the GFP reporter gene.

FIG. 20. Reprogramming of human fibroblasts into iPSCs. Left: 2-day-old human foreskin fibroblast cells (HFF; ATCC-CRL-2097) induced with Oct4/Klf4/Sox2/c-Myc-lentivirus. Right: 28-day-old iPSC colony.

FIG. 21. PTB competes with the essential splicing factor, U2AF65, for binding to the intronic cis-splicing element.

### DETAILED DESCRIPTION OF THE INVENTION

In accordance with the present invention there may be employed conventional molecular biology, microbiology, immunology, and recombinant DNA techniques within the skill of the art. Such techniques are explained fully in the literature. See, e.g., Sambrook et al, “Molecular Cloning: A Laboratory Manual” (3^{rd} edition, 2001); “Current Protocols in Molecular Biology” Volumes I-III [Ausubel, R. M., ed. (1999 and updated bimonthly)]; “Cell Biology: A Laboratory Handbook” Volumes I-III [J. E. Celis, ed. (1994)]; “Current Protocols in Immunology” Volumes I-IV [Coligan, J. E., ed. (1999 and updated bimonthly)]; “Oligonucleotide Synthesis” (M. J. Gait ed. 1984); “Nucleic Acid Hybridization” [B. D. Hames & S. J. Higgins eds. (1985)]; “Transcription And Translation” [B. D. Hames & S. J. Higgins, eds. (1984)]; “Culture of Animal Cells, 4^{th} edition” [R. I. Freshney, ed. (2000)]; “Immobilized Cells And Enzymes” [IRL Press, (1986)]; B. Perbal, “A Practical Guide To Molecular Cloning” (1988); Using Antibodies: A Laboratory Manual: Portable Protocol No. I, Harlow, Ed and Lane, David (Cold Spring Harbor Press, 1998); Using Antibodies: A Laboratory Manual, Harlow, Ed and Lane, David (Cold Spring Harbor Press, 1999).

Therefore, if appearing herein, the following terms shall have the definitions set out below.

The term “primer” as used herein refers to an oligonucleotide, whether occurring naturally as in a purified restriction digest or produced synthetically, which is capable of acting as a point of initiation of synthesis when placed under conditions in which synthesis of a primer extension product, which is complementary to a nucleic acid strand, is induced, i.e., in the presence of nucleotides and an inducing agent such as a DNA polymerase and at a suitable temperature and pH. The primer may be either single-stranded or double-stranded and must be sufficiently long to prime the synthesis of the desired extension product in the presence of the inducing agent. The exact length of the primer depends upon many factors, including temperature, source of primer and use of the method. For example, for diagnostic applications, depending on the complexity of the target sequence, the oligonucleotide primer typically contains 15-25 or more nucleotides, although it may contain fewer nucleotides.

The primers herein are selected to be “substantially” complementary to different strands of a particular target DNA sequence. This means that the primers must be sufficiently complementary to hybridize with their respective strands. Therefore, the primer sequence need not reflect the exact sequence of the template. For example, a non-complementary nucleotide fragment may be attached to the 5′ end of the primer, with the remainder of the primer sequence being complementary to the strand. Alternatively, non-complementary bases or longer sequences can be interspersed into the primer, provided that the primer sequence has sufficient complementarity with the sequence of the strand to hybridize therewith and thereby form the template for the synthesis of the extension product.

Two DNA sequences are “substantially homologous” when at least about 75% (preferably at least about 80%, and most preferably at least about 90 or 95%) of the nucleotides match over the defined length of the DNA sequences. Sequences that are substantially homologous can be identified by comparing the sequences using standard software available in sequence data banks, or in a Southern hybridization experiment under, for example, stringent conditions as defined for that particular system. Defining appropriate hybridization conditions is within the skill of the art. See, e.g., Maniatis et al., supra; DNA Cloning, Vols. I & II, supra; Nucleic Acid Hybridization, supra.

Two amino acid sequences are “substantially homologous” when at least about 70% of the amino acid residues (preferably at least about 80%, and most preferably at least about 90 or 95%) are identical, or represent conservative substitutions.

The term “standard hybridization conditions” refers to salt and temperature conditions substantially equivalent to 5×SSC and 65° C. for both hybridization and wash. However, one skilled in the art will appreciate that such “standard hybridization conditions” are dependent on particular conditions including the concentration of sodium and magnesium in the buffer, nucleotide sequence length and concentration, percent mismatch, percent formamide, and the like. Also important in the determination of “standard hybridization conditions” is whether the two sequences hybridizing are RNA-RNA, DNA-DNA or RNA-DNA. Such standard hybridization conditions are easily determined by one skilled in the art according to well known formulae, wherein hybridization is typically 10-20° C. below the predicted or determined Tₘ with washes of higher stringency, if desired.

A “splicing reporter construct” refers to any DNA construct that replicates the alternative splicing pattern of an alternatively spliced transcript. The term “splicing reporter construct” is meant to include a DNA construct that includes, but is not limited to, a reporter gene, a promoter to drive expression, and sequences from the splice regions of the alternatively spliced transcript.

An “antibody” is any immunoglobulin, including antibodies and fragments thereof, that binds a specific epitope. The term encompasses polyclonal, monoclonal, and chimeric antibodies, the last mentioned described in further detail in U.S. Pat. Nos. 4,816,397 and 4,816,567.

The general methodology for making monoclonal antibodies by hybridomas is well known. Immortal, antibody-producing cell lines can also be created by techniques other than fusion, such as direct transformation of B lymphocytes with oncogenic DNA, or transfection with Epstein-Barr virus. See, e.g., M. Schreier et al., “Hybridoma Techniques” (1980); Hammerling et al., “Monoclonal Antibodies And T-cell Hybridomas” (1981); Kennett et al., “Monoclonal Antibodies” (1980); see also U.S. Pat. Nos. 4,341,761; 4,399,121; 4,427,783; 4,444,887; 4,451,570; 4,466,917; 4,472,500; 4,491,632; 4,493,890. See also, Niman et al., Proc. Natl. Acad. Sci. USA, 80:4949-4953 (1983). Typically, the peptide or polypeptide is used either alone or conjugated to an immunogenic carrier. The hybridomas are screened for the ability to produce an antibody that immunoreacts with the peptide or polypeptide of interest.

Methods for producing polyclonal anti-polypeptide antibodies are well-known in the art. See U.S. Pat. No. 4,493,795 to Nestor et al. A monoclonal antibody, typically containing Fab and/or F(ab′)₂ portions of useful antibody molecules, can be prepared using the hybridoma technology described in Antibodies—A Laboratory Manual, Harlow and Lane, eds., Cold Spring Harbor Laboratory, New York (1988), which is incorporated herein by reference. Briefly, to form the hybridoma from which the monoclonal antibody composition is produced, a myeloma or other self-perpetuating cell line is fused with lymphocytes obtained from the spleen of a mammal hyperimmunized with a peptide or polypeptide of interest.

Splenocytes are typically fused with myeloma cells using polyethylene glycol (PEG) 6000. Fused hybrids are selected by their sensitivity to HAT. Hybridomas producing a monoclonal antibody useful in practicing this invention are identified by their ability to immunoreact with the peptide or polypeptide of interest.

A monoclonal antibody useful in practicing the present invention can be produced by initiating a monoclonal hybridoma culture comprising a nutrient medium containing a hybridoma that secretes antibody molecules of the appropriate antigen specificity. The culture is maintained under conditions and for a time period sufficient for the hybridoma to secrete the antibody molecules into the medium. The antibody-containing medium is then collected. The antibody molecules can then be further isolated by well-known techniques.

Media useful for the preparation of these compositions are both well-known in the art and commercially available and include synthetic culture media, inbred mice and the like. An exemplary synthetic medium is Dulbecco's minimal essential medium (DMEM; Dulbecco et al., Virol. 8:396 (1959)) supplemented with 4.5 gm/l glucose, 20 mm glutamine, and 20% fetal calf serum. An exemplary inbred mouse strain is the Balb/c.

An antibody used in the methods of this invention may be an affinity purified polyclonal antibody or a monoclonal antibody (mAb), and may be used in the form of Fab, Fab′, F(ab′)₂ or F(v) portions of whole antibody molecules.

An “antibody combining site” is that structural portion of an antibody molecule comprised of heavy and light chain variable and hypervariable regions that specifically binds antigen.

The phrase “antibody molecule” in its various grammatical forms as used herein contemplates both an intact immunoglobulin molecule and an immunologically active portion of an immunoglobulin molecule.

Exemplary antibody molecules are intact immunoglobulin molecules, substantially intact immunoglobulin molecules and those portions of an immunoglobulin molecule that contains the paratope, including those portions known in the art as Fab, Fab′, F(ab′)₂ and F(v), which portions are preferred for use in the therapeutic methods described herein.

Fab and F(ab′)₂ portions of antibody molecules are prepared by the proteolytic reaction of papain and pepsin, respectively, on substantially intact antibody molecules by methods that are well-known. See for example, U.S. Pat. No. 4,342,566 to Theofilopolous et al. Fab′ antibody molecule portions are also well-known and are produced from F(ab′)₂ portions followed by reduction of the disulfide bonds linking the two heavy chain portions as with mercaptoethanol, and followed by alkylation of the resulting protein mercaptan with a reagent such as iodoacetamide. An antibody containing intact antibody molecules is preferred herein.

The phrase “monoclonal antibody” in its various grammatical forms refers to an antibody having only one species of antibody combining site capable of immunoreacting with a particular antigen. A monoclonal antibody thus typically displays a single binding affinity for any antigen with which it immunoreacts. A monoclonal antibody may therefore contain an antibody molecule having a plurality of antibody combining sites, each immunospecific for a different antigen; e.g., a bispecific (chimeric) monoclonal antibody.

The presence of the protein product of an alternatively spliced transcript in cells can be ascertained by the usual immunological procedures applicable to such determinations. A number of useful procedures are known. Three such procedures which are especially useful utilize antibody Ab₁ labeled with a detectable label, or antibody Ab₂ labeled with a detectable label.

It will be seen from the above, that a characteristic property of Ab₂ is that it will react with Ab₁. This is because Ab₁ raised in one mammalian species has been used in another species as an antigen to raise the antibody Ab₂. For example, Ab₂ may be raised in goats using rabbit antibodies as antigens. Ab₂ therefore would be anti-rabbit antibody raised in goats. For purposes of this description and claims, Ab₁ will be referred to as a primary antibody, and Ab₂ will be referred to as a secondary or anti-Ab₁ antibody.

The labels most commonly employed for these studies are radioactive elements, enzymes, chemicals which fluoresce when exposed to ultraviolet light, and others.

A number of fluorescent materials are known and can be utilized as labels. These include, for example, fluorescein, rhodamine, auramine, Texas Red, AMCA blue and Lucifer Yellow. A particular detecting material is anti-rabbit antibody prepared in goats and conjugated with fluorescein through an isothiocyanate.

Enzyme labels are likewise useful, and can be detected by any of the presently utilized colorimetric, spectrophotometric, fluorospectrophotometric, amperometric or gasometric techniques. The enzyme is conjugated to the selected particle by reaction with bridging molecules such as carbodiimides, diisocyanates, glutaraldehyde and the like. Many enzymes which can be used in these procedures are known and can be utilized. The preferred are peroxidase, B-glucuronidase, B-D-glucosidase, B-D-galactosidase, urease, glucose oxidase plus peroxidase and alkaline phosphatase. U.S. Pat. Nos. 3,654,090; 3,850,752; and 4,016,043 are referred to by way of example for their disclosure of alternate labeling material and methods.

The invention provides a method of distinguishing a PSC from a SDC by identifying in the said cell the presence of an alternatively spliced transcript, which is preferentially expressed in said PSC compared to said SDC. The alternatively spliced transcript may be unique to the PSC, or may be expressed at a higher level in the PSC compared to the SDC. In general, an alternatively spliced transcript may be expressed at a 25-35% higher level than in the SDC, more preferably 50-70% higher level than in the SDC, and most preferably 100% or higher level than in the SDC in order to be considered a useful reagent for use in accordance with the methods of the invention.

The alternatively spliced transcript is preferably an exon-included transcript, but may also be an exon-excluded transcript.

Preferred exon-included transcripts include those expressed from a nucleotide binding protein 2 (NUB2), a nucleoside diphosphate kinase A (NDKA), a purinergic receptor P2X, ligand-gated ion channel 5 (P2RX5), or a DNA cytosine-5-methyltransferase 3 beta (DNMT3B) gene. Particularly preferable are alternatively spliced transcripts expressed from the DNMT3B gene. Most preferable are alternatively spliced transcripts which include exon 10 of the DNMT3B gene.

Preferred exon-excluded transcripts include those expressed from a feline sarcoma oncogene (FES), a cell division cycle 25 homolog A (CDC25A), or a tyrosine kinase 2 (TYK2) gene.

Any method for detecting the alternatively spliced transcript can be used, but preferably one uses either a nucleic acid that binds, or a set of primers that amplify said alternatively spliced transcript. Such amplification can be performed by any method known in the art, but reverse transcription polymerase chain reaction (RT-PCR) and real time PCR, including qualitative real-time PCR (qRT-PCR), are among the preferred methods.

Additional methods for detecting the presence of the alternatively spliced transcript are provided herein. These include the use of splicing reporter constructs that incorporate the splicing mechanism of the alternatively spliced transcript. The construct includes reporter genes known to those of skill in the art. These may include but are not limited to, GFP, RFP, luciferase or derivatives thereof. More preferably, the reporter gene is GFP. Additionally, the construct contains a promoter to drive expression of the construct. Various promoters are well-known in the art for use in expression and reporter constructs, and it is contemplated that this aspect of the invention may incorporate any of said promoters. One particularly preferred embodiment includes the CMV promoter. The construct also includes canonical splice sites arranged such that the PSC-specific splicing pattern will result in an in-frame transcript resulting in reporter gene translation. However, if a splice event other than the PSC-specific splicing event occurs, this will result in an out-of-frame transcript which would prevent the correct translation of the reporter gene. It is contemplated that this approach may be used for any gene that is preferentially expressed in PSC's as compared to SDC's. In a preferred embodiment of this aspect of the invention, the splice sites of DNMT3B can be used. In a particularly preferred embodiment, the splice sites of DNMT3Be10 can be used. In a most preferred embodiment, the CMV promoter and an ATG start codon are fused 5′ to the DNMT3B sequence, which comprises the DNMT3B exon 10, the exon 10 splice sites, and an appropriate amount of flanking intronic sequence from introns 9/10 and 10/11, and exon 11, and wherein a GFP reporter gene is fused to the 3′ end of the DNMT3B sequences.

Any method for detecting the reporter gene expression in cells en masse and separating expressing cells from non-expressing cells may be employed. In a preferred embodiment, flow cytometry is used.

The invention also provides a method of distinguishing a PSC from a SDC, by identifying in the cell the presence of a polypeptide encoded by the alternatively spliced transcript. The polypeptide may be unique to the PSC, or may be expressed at a higher level in the PSC compared to the SDC.

Any polypeptide produced by an alternatively spliced transcript (which is preferentially expressed in the PSC) can be used, but preferred polypeptides are encoded by a nucleotide binding protein 2 (NUB2), a nucleoside diphosphate kinase A (NDKA), a purinergic receptor P2X, ligand-gated ion channel 5 (P2RX5), or a DNA cytosine-5-methyltransferase 3 beta (DNMT3B) gene, and most preferably the DNMT3B gene, and most preferably by alternatively spliced DNMT3B transcripts which include exon 10. Preferably, the polypeptide includes at least an immunostimulatory portion of the sequence: KSKVRRAGSRKLESR such that antibodies to the polypeptide can be generated. Most preferably, the antibody is SG1, which is capable of detecting a single DNMT3B protein isoform expressed in PSC's and not in SDC's.

The antibody may be polyclonal or monoclonal, but is preferably at least partially purified. The antibody may be an immunoreactive portion of an antibody, may be recombinantly produced, or may be chimeric or humanized. The antibody may be detectably labeled, or may be visualized using a labeled secondary antibody.

It is contemplated that additional assays may be employed, either alone or in combination with the methods detailed above, to evaluate and compare the level of genomic instability in PSC's and SDC's. These include DAPI staining, tubulin antibodies, or any other comparable reagent suitable for the evaluation of genomic instability.

In yet more detail, the present invention is described by the following items which represent additional embodiments hereof.

1. A method of distinguishing a pluripotent stem cell (PSC) from a spontaneously differentiated cell (SDC), comprising identifying in said cell the presence of an alternatively spliced transcript which is preferentially expressed in said PSC compared to said SDC.

2. The method of item 1, wherein the alternatively spliced transcript is unique to the PSC.

3. The method of item 1, wherein the alternatively spliced transcript is expressed at a higher level in the PSC compared to the SDC.

4. The method of item 1, wherein the alternatively spliced transcript is an exon-included transcript.

5. The method of item 1, wherein the alternatively spliced transcript is an exon-excluded transcript.

6. The method of item 1, wherein the alternatively spliced transcript is expressed from a nucleotide binding protein 2 (NUB2), a nucleoside diphosphate kinase A (NDKA), a purinergic receptor P2X, ligand-gated ion channel 5 (P2RX5), or a DNA cytosine-5-methyltransferase 3 beta (DNMT3B) gene.

7. The method of item 6, wherein the alternatively spliced transcript is expressed from the DNMT3B gene.

8. The method of item 7, wherein the alternatively spliced transcript includes exon 10 of the DNMT3B gene.

9. The method of item 8, wherein the alternatively spliced transcript includes the nucleotide sequence:

AAGUCGAAGGUGCGUCGUGCAGGCAGUAGGAAAUUAGAAUCAAGG.

10. The method of item 1, wherein the identifying is performed using a nucleic acid that binds the alternatively spliced transcript.

11. The method of item 1, wherein the identifying is performed using primers that amplify the alternatively spliced transcript.

12. The method of item 11, wherein the amplifying is performed by reverse transcription polymerase chain reaction (RT-PCR).

13. The method of item 11, wherein the amplifying is performed by real time PCR.

14. A method of distinguishing a pluripotent stem cell (PSC) from a spontaneously differentiated cell (SDC), including identifying in said cell the presence of a polypeptide encoded by an alternatively spliced transcript which is preferentially expressed in the PSC compared to the SDC.

15. The method of item 14, wherein the polypeptide is unique to the PSC.

16. The method of item 14, wherein the polypeptide is expressed at a higher level in the PSC compared to the SDC.

17. The method of item 14, wherein the polypeptide is encoded by a nucleotide binding protein 2 (NUB2), a nucleoside diphosphate kinase A (NDKA), a purinergic receptor P2X, ligand-gated ion channel 5 (P2RX5), or a DNA cytosine-5-methyltransferase 3 beta (DNMT3B) gene.

18. The method of item 17, wherein the polypeptide is encoded by the DNMT3B gene.

19. The method of item 18, wherein the polypeptide is encoded by exon 10 of the DNMT3B gene.

20. The method of item 19, wherein the polypeptide includes the sequence: KSKVRRAGSRKLESR.

21. The method of item 14, wherein the identifying is performed using an antibody which binds the polypeptide.

22. The method of item 21, wherein the antibody is a polyclonal antibody.

23. The method of item 21, wherein the antibody is a monoclonal antibody.

24. An antibody to a polypeptide encoded by DNMT3B exon 10.

25. The antibody of item 24, wherein the antibody binds the polypeptide sequence: KSKVRRAGSRKLESR.

26. The antibody of item 24, wherein the antibody is a polyclonal antibody.

27. The antibody of item 24, wherein the antibody is a monoclonal antibody.

28. The antibody of item 26, wherein the antibody is SG1.

29. The antibody of item 24, wherein the antibody is detectably labeled.

30. The method of item 1, wherein the alternatively spliced transcript is identified using a reporter gene construct.

31. The method of item 14, wherein the reporter gene construct comprises: a promoter; a start codon; DNMT3Be10 sequence containing splice sites and intronic and exonic sequences; and a reporter gene.

32. The method of item 15, wherein the promoter is a CMV promoter.

33. The method of item 15, wherein the DNMT3Be10 sequence includes the 5′ splice site of intron 9/10; intron 9/10; the 3′ splice site between intron 9/10 and exon 10; the 5′ splice site between exon 10 and intron 10/11; the 3′ splice site between intron 10/11 and exon 11; and exon 11.

34. The method of item 15 wherein the reporter gene is Green Fluorescent Protein (GFP).

The compositions and processes of the present invention will be better understood in connection with the following examples, which are intended as an illustration only and not limiting of the scope of the invention. Various changes and modifications to the disclosed embodiments will be apparent to those skilled in the art and such changes and modifications including, without limitation, those relating to the processes, formulations and/or methods of the invention may be made without departing from the spirit of the invention and the scope of the appended claims.

### EXAMPLES

### Example 1

For measuring genome wide changes in expression levels exon microarrays can be used because probesets in cDNA arrays tend to be clustered near 3′ untranslated regions and, therefore, cannot distinguish alternatively splice variants. In contrast, probesets in exon arrays span the gene and tend to give more reproducible results when examining changes in whole gene expression levels. For bioinformatic analysis of exon microarray data, Partek Genome Suite applications can be used (Gopalakrishna-Pillai and Iverson, 2010).

Newer cDNA microarrays containing gene-spanning probesets have been developed. The Affymetrix Gene 1.0 ST Array uses a subset of probes from the Affymetrix Exon 1.0 ST Array and gives better gene coverage than standard cDNA arrays. However, both the Exon 1.0 ST Array and the Gene 1.0 ST Array cover only well-annotated transcripts, and are often missing probesets for less well-annotated alternatively spliced transcripts. In particular, the Exon 1.0 ST Array and the Gene 1.0 ST Array are both missing probesets recognizing DNMT3B exon 10. When characterizing DNA methylation patterns in hESCs and iPSCs generated from cells of all three lineages, microarray analysis was used to determine expression levels of DNA methyltransferases, including DNMT1, DNMT3L, DNMT3A and DNMT3B (Ohi et al., 2011). Since no significant differences in expression levels were observed, the authors concluded that differential expression of DNMTs played no role in incomplete DNA methylation underlying epigenetic memory. However, Affymetrix Gene 1.0 ST Arrays were used for this gene expression profiling, and—as stated above—do not contain DNMT3B exon 10 probesets.

For this reason the approach relied on the use of both exon microarrays and RNAseq analyses (Sultan et al., 2008; Trapnell et al., 2010). RNAseq analysis does not suffer from the same limitations as microarrays because it is not dependent on any a priori knowledge of well-annotated splice variants or on previously defined exon/intron boundaries. It is for this reason that both Affymetrix Exon 1.0 ST Arrays and RNAseq analysis were used to initially identify alternatively spliced, protein-coding exons uniquely expressed in pluripotent stem cells (PCSs) and absent from spontaneously differentiated cells (SDCs). Several candidate genes/transcripts exhibiting alternative splicing as pluripotent hESCs transition to differentiated states were selected and subjected to RT-PCR analysis for subsequent validation (Gopalakrishna-Pillai and Iverson, 2011).

Using the methods detailed above, the inventors have surprisingly found that the exon 1—included alternatively spliced variant of DNMT3B (DNMT3Be10) appears to be the sole ‘exon-included’ splice variant expressed exclusively in hESCs, indicating that this domain is both unique to, and characteristic of, a normal pluripotent cell (Gopalakrishna-Pillai and Iverson, 2011). The human DNMT3B gene encodes as many as 40 different isoforms through alternative splicing of DNMT3B transcripts. Various DNMT3B splice isoforms are highly expressed in the human female germ line, preimplantation embryos, and embryonic stem cells, and are differentially expressed during development and tumorigenesis (Linhart et al, 2007; Beyrouthy et al, 2009; Gopalakrishnan et al, 2009). DNMT3B was previously identified as a commonly overexpressed marker of 59 hESC lines by microarray analysis (Adewumi et al, 2007). However, uniquely expressed splice variants are not generally detectable using conventional cDNA microarrays. DNMT3B was also suggested to be a specific marker of bona fide human pluripotent stem cells (Chan et al, 2009) based on qRT-PCR analysis that did demonstrate a high degree of specificity of expression of DNMT3B transcripts in PSCs relative to partially reprogrammed cells. However, not all DNMT3B transcripts or DNMT3B protein isoforms are unique and reliable markers of pluripotent stem cells.

Upon induction of pluripotency, gene expression profiling indicates that DNMT3Be10 exhibits robust up-regulation and increased DNMT3Be10 expression correlates with a greater degree of ‘pluripotency’ as confirmed by teratoma formation (Chan et al., 2009). This finding, combined with evidence that ‘somatic memory’ arises from incomplete DNA methylation (Ohi et al., 2011), suggests that DNMT3Be10 plays a role in reestablishing DNA methylation patterns characteristic of pluripotent cells.

Organization of DNA into higher order chromatin structures has profound effects on gene expression. Mutations in a number of genes are associated with human ‘chromatin’ disorders such as Rett and ICF syndrome. ICF syndrome is a rare autosomal recessive disease characterized by severe immunodeficiency and marked genomic instability resulting from hypomethylation of pericentric heterochromatin that results in mitotic defects (Ehrlich et al., 2006). About 60% of ICF patients carry mutations in DNMT3B, which tend to cluster in the C-terminal catalytic domain. However, DNMT3B also exhibits a transcriptional repressor function, which maps to the central region of the protein (in close proximity to the exon 10 encoded domain), and is independent of the methyltransferase domain (Matarazzo et al., 2009). Werner Syndrome is an autosomal recessive disorder caused by mutations in the WRN gene and is characterized by premature aging and aberrant DNA repair (Chen et al., 2003a; Turaga et al., 2007). WRN protein (WRNp) acts to recruit DNMT3B to the OCT4 promoter, suggesting that WRNp and DNMT3B may play a role in the stem cell pluripotency/differentiation switch by modulating OCT4 transcription (Smith et al., 2010).

Because DNMT3B is a de novo DNA methyltransferase required for transcriptional repression (Okano et al., 1999), loss of DNMT3B results in embryonic lethality, indicating its critical role in normal development (Bachman et al., 2001). DNMT3B mutations result in immunodeficiency, centromeric instability, facial anomalies (ICF) syndrome characterized by aberrant DNA methylation and genomic instability (Matarazzo et al., 2009). In addition to DNMT3B, DNMT3A is also a major de novo DNA methyltransferase. Loss of one or both results in abnormal global DNA methylation patterns. However, loss of DNMT3B (unlike DNMT3A) also results in hypomethylation of centromeric and pericentromeric satellite regions that leads to centromeric instability and mitotic defects (Hansen et al, 1999). Although the precise function of the DNMT3B exon 10-encoded peptide remains unknown, it lies between the PWWP and the ring-type zinc finger domains suggesting that it may play a role in modulating protein-protein interactions important for DNMT3B binding to H4K20me and/or targeting of DNMT3B to particular chromosomal sites (Weisenberger et al, 2004; Chen et al, 2004). A series of recent reports indicate that gene expression profiles of iPSCs and hESCs are non-identical and that some iPSCs retain an epigenetic memory of their cell type of origin that could arise from distinct global and/or gene-specific DNA methylation patterns (Chin et al, 2009; Chin et al, 2010; Deng et al, 2009; Doi et al, 2009, Guenther et al, 2010; Newman and Cooper, 2010; Kim et al, 2010). Furthermore, recent evidence indicates that the Werner Syndrome gene product, WRNp, localizes to the OCT4 promoter of human PSCs undergoing retinoic acid induced differentiation where it plays a role in de novo DNA methylation by recruiting DNMT3B to the OCT4 promoter (Smith et al, 2010). While not desiring to be bound by a particular theory, proteins encoded by DNMT3B exon 10-containing transcripts may play a crucial role in establishing de novo DNA methylation patterns that are characteristic of the pluripotent state perhaps by regulating transcription of the pluripotency transcription factor, OCT4, and, in so doing, might affect the efficiency and/or stability of nuclear reprogrammed iPSCs. Finally, the previously noted similarities in pluripotent and cancer stem cell gene expression patterns (Clarke and Fuller, 2006) suggest that DNMT3B exon 10 may be a specific biomarker of the stem cell component of some tumors. The restricted expression of DNMT3Be10, combined with known deleterious effects of DNMT3B mutations and other splice variants on embryonic development, genome stability and in tumorigenesis, makes DNMT3Be10 a prime candidate for a gene that plays an essential role in stem cell self-renewal, while concomitantly preserving the integrity of the genome required of a normal hESC. It is not unreasonable to hypothesize that the DNMT3Be10 splice variant may operate, directly or indirectly, to regulate both the switch between self-renewal and differentiation in hESCs and to facilitate reprogramming in iPSCs. Determination of the complex mechanisms regulating the stem cell self-renewal/differentiation switch is crucial for understanding normal development, for identifying other genes involved in this process, for devising strategies to create normal cells suitable for therapeutic applications, and for developing reagents useful for distinguishing between PSC's and SDC's.

DNMT3B and DNMT3A are both expressed in pluripotent stem cells. Although they have different DNA methylation consensus sequences, they have both common and distinct DNA targets and, interestingly, interact with different transcription factors to effect site-specific DNA methylation (Chen et al., 2003b; Hervouet et al., 2009). DNMT3A and DNMT3B cooperate in initial targeting of de novo DNA methylation to the OCT4 promoter in hESCs undergoing differentiation, but DNMT3B is not required for completion of this process (Athanasiadou et al., 2010). Loss of DNMT3A was recently shown to result in over-expression of hematopoietic stem cell (HSC) ‘multipotency’ genes and down regulation of ‘differentiation’ genes, indicating that DNMT3A plays a critical role in epigenetic silencing of HSC regulatory genes, and thereby promotes efficient differentiation of HSCs (Challen et al., 2012).

Epigenetic modification of DNA via methylation of CpG islands in 5′ regulatory regions has long been associated with changes in gene expression levels. Recent evidence demonstrates that histone modifications precede DNA methylation indicating that modification to the underlying histone code is a more reliable indicator of stable epigenetic changes (Rada-Iglesias and Wysocka, 2011). Thus, it is becoming increasingly clear that 5meCpG may be a surrogate marker for underlying histone modifications. Decreases in DNA methylation at CpG islands are often associated with loss of ‘repressive’ histone modifications such as H3K27me3 and gains in ‘active’ H3K4me3, but many genes showing changes in methylation status of CpG islands do not show consistent changes in bivalent chromatin modifications. This may be particularly true of DNMT3B-mediated de novo DNA methylation; only a subset of down-regulated genes in ICF patients identified by microarray analysis, validated by RT-PCR, and harboring 5′ proximal methylation of CpG islands exhibited bivalent chromatin modifications (Jin et al., 2008). The ‘processive’ nature of the DNMT3B enzyme tends to accelerate methylation at CpG rich sites (Gowher and Jeltsch, 2002), which can lead to wide spread DNA methylation that may (or may not) accurately reflect bivalent chromatin modifications that result in switching from transcriptionally ‘repressed’ to ‘active’ states.

DNMT3B is known to interact with a wide variety of nuclear proteins including the RecQ DNA helicase, WRNp (Smith et al., 2010), the transcription factor, SP1 (Hervouet et al., 2009), and the centromere protein, CENP-C (Gopalakrishnan et al., 2009a). Although domain mapping experiments indicate that CENP-C interacts with DNMT3B through its N-terminal domain, the proximity of the DNMT3Be10-encoded domain to the centrally located PWWP domain, which is required for chromatin targeting (Chen et al., 2004; Ge et al., 2004), suggests the DNMT3Be10-encoded domain may also play a role in targeting DNMT3B to particular chromatin sites. For example, an evolutionarily conserved POU5F1 (OCT4) binding site is linked to a SP1 binding site within the 5′ regulatory region of the FZD5 promoter (Katoh and Katoh, 2007), suggesting DNMT3B is directly involved in regulating expression of FZD5 in hESCs via OCT4 and SP1. SP1 binding sites are also seen in the 5′ regulatory region of the FZD7 promoter (Katoh, 2007).

Several genes encoding proteins involved in important signaling pathways were screened to detect alternatively spliced transcripts that exhibited differential expression in pluripotent stem cells (PSCs) relative to spontaneously differentiated cells (SDCs). Transcripts containing the alternatively spliced exon 10 of the de novo DNA methyltransferase gene, DNMT3B, were identified that are expressed in PSCs. To demonstrate the utility and superiority of splice variant specific reagents for stem cell research, a peptide encoded by DNMT3B exon 10 was used to generate an antibody, SG1. The SG1 antibody detects a single DNMT3B protein isoform that is expressed only in PSCs but not in SDCs. The SG1 antibody is also demonstrably superior to other antibodies at distinguishing PSCs from SDCs in mixed cultures containing both pluripotent stem cells and partially differentiated derivatives. The tightly controlled down regulation of DNMT3B exon 10 containing transcripts (and exon 10 encoded peptide) upon spontaneous differentiation of PSCs suggests that this DNMT3B splice isoform is characteristic of the pluripotent state. Alternatively spliced exons, and the proteins they encode, represent a vast untapped reservoir of novel biomarkers that can be used to develop superior reagents for stem cell research and to gain further insight into mechanisms controlling stem cell pluripotency.

### Materials and Methods

### Pluripotent Stem and Differentiated Cell Culture

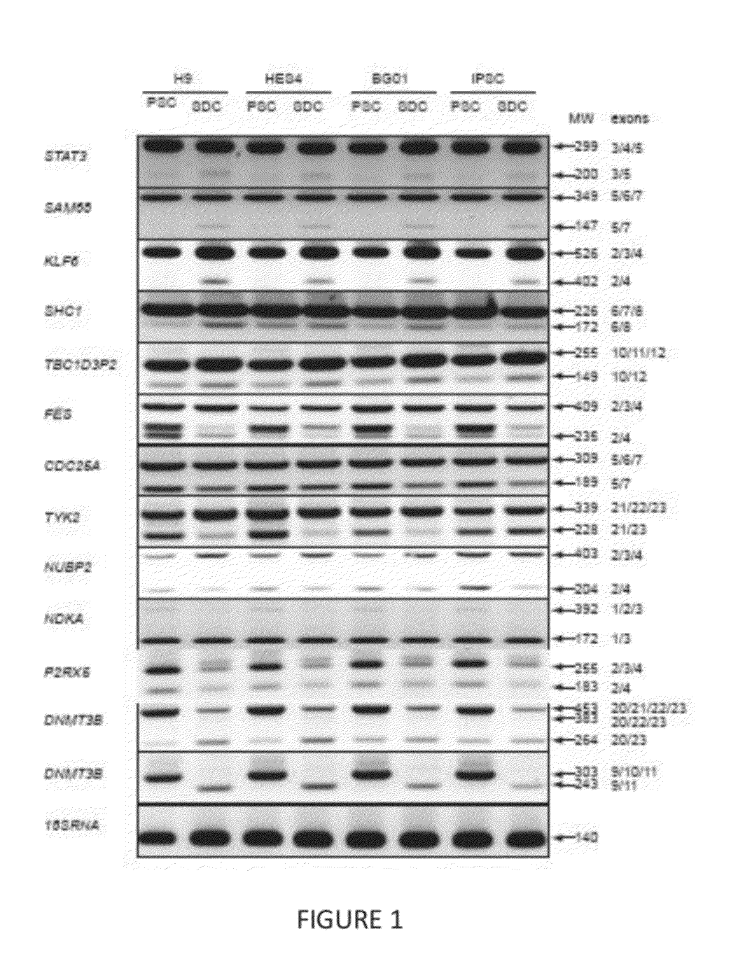

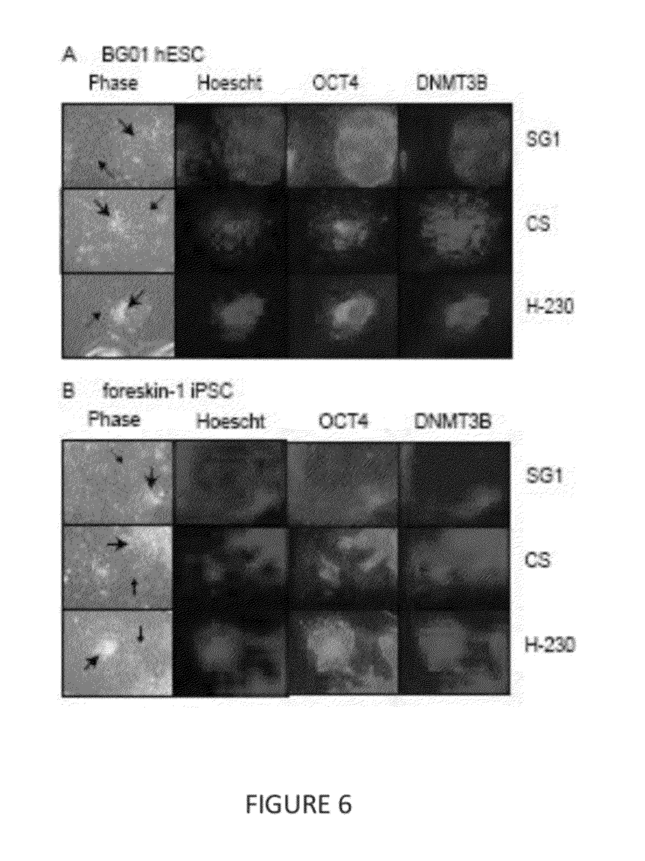

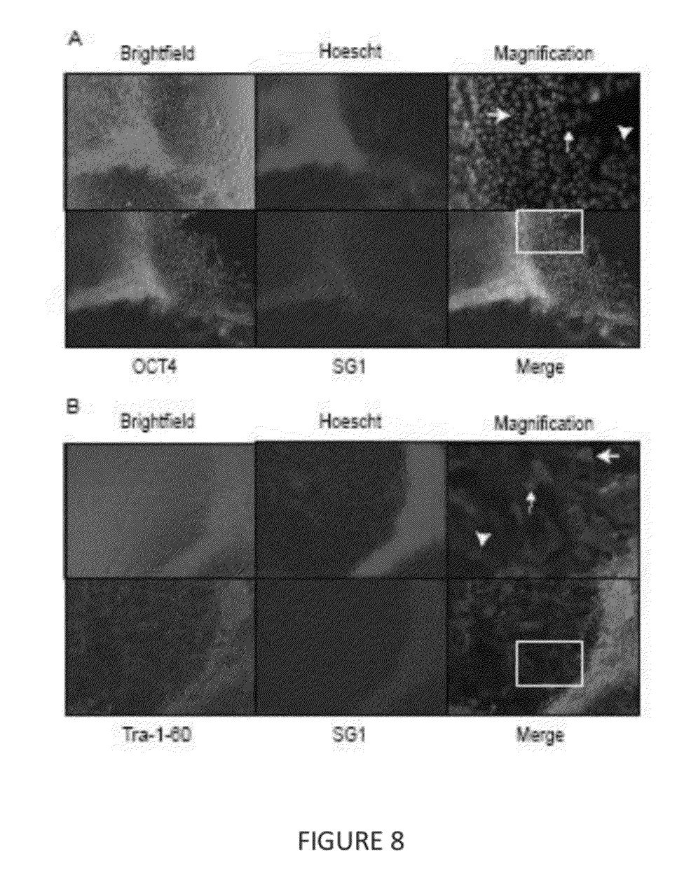

Karyotypically normal PSCs, including three hESC lines (H9 [WiCell], HES4 [IS], BG01 [Bresagen]) and the iPSC foreskin clone 1 (a generous gift from Dr. James Thomson,), were maintained either on gamma-irradiated mouse embryonic fibroblasts feeder layer (CF-1, ATCC) or under feeder-independent conditions on matrigel coated dishes (BD) as described in detail previously (Gopalakrishna-Pillai and Iverson, 2010) and briefly below. The hESCs were expanded on matrigel prior to harvesting RNA and protein to prevent any contamination from MEF-derived mouse gene products in molecular experiments. Media contained DMEM/F-12 with glutamine, 20% knockout serum replacement, 2 mM non-essential amino acids (all from Invitrogen) and 20 ng/ml zbFGF (Ludwig et al, 2006). Cells were cultured under 5% CO₂ at 37° C. For passaging, 5-6 day old hESC colonies were cut into small pieces (100-200 cells) by mechanical dissection using a 27G hypodermic needle and transferred to new dishes at a split ratio of 1:3. To obtain spontaneously differentiated cells, undifferentiated PSC colonies grown on matrigel were fed with hESC media without zbFGF for the number of days indicated in each figure legend. Specifically, mixed cultures of PSCs and SDCs were produced by culturing in the absence of zbFGF for 4-5 days (FIGS. 6 and 8), while relatively homogeneous cultures of SDCs were obtained by maintaining in culture minus zbFGF for 14-15 days (FIGS. 1, 2, and 5). Cultures of homogeneous SDCs (14-15 days minus zbFGF) were examined for any clusters of undifferentiated cells, which were removed from the dish prior to harvesting RNA or protein for molecular experiments.

### Semi-Quantitative and Realtime RT-PCR Analysis

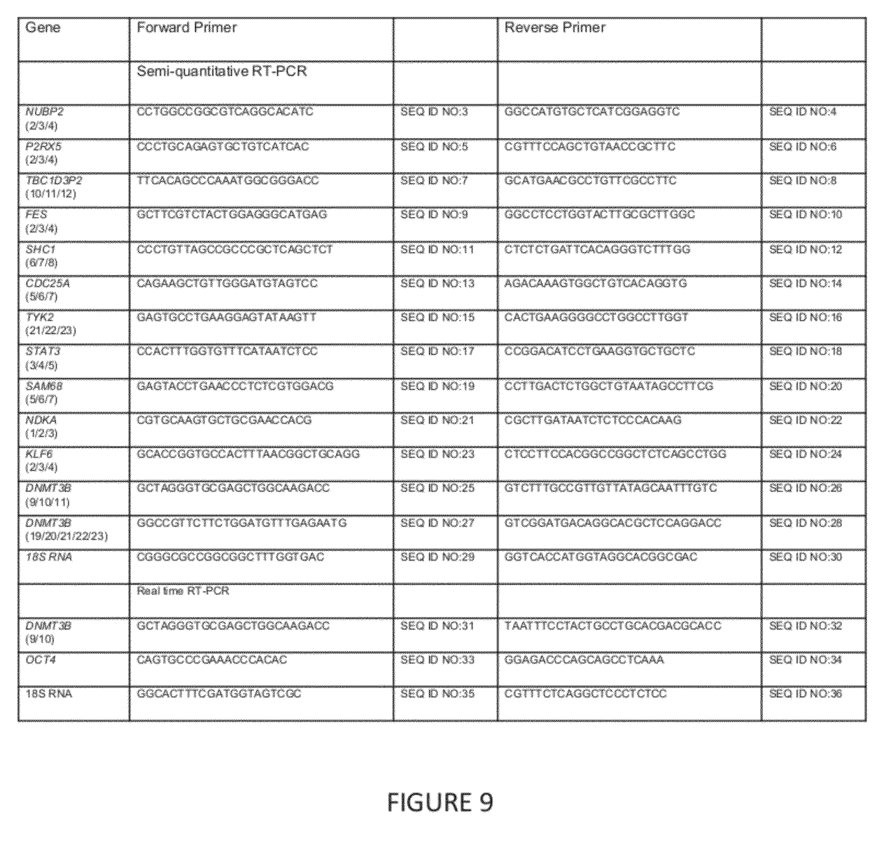

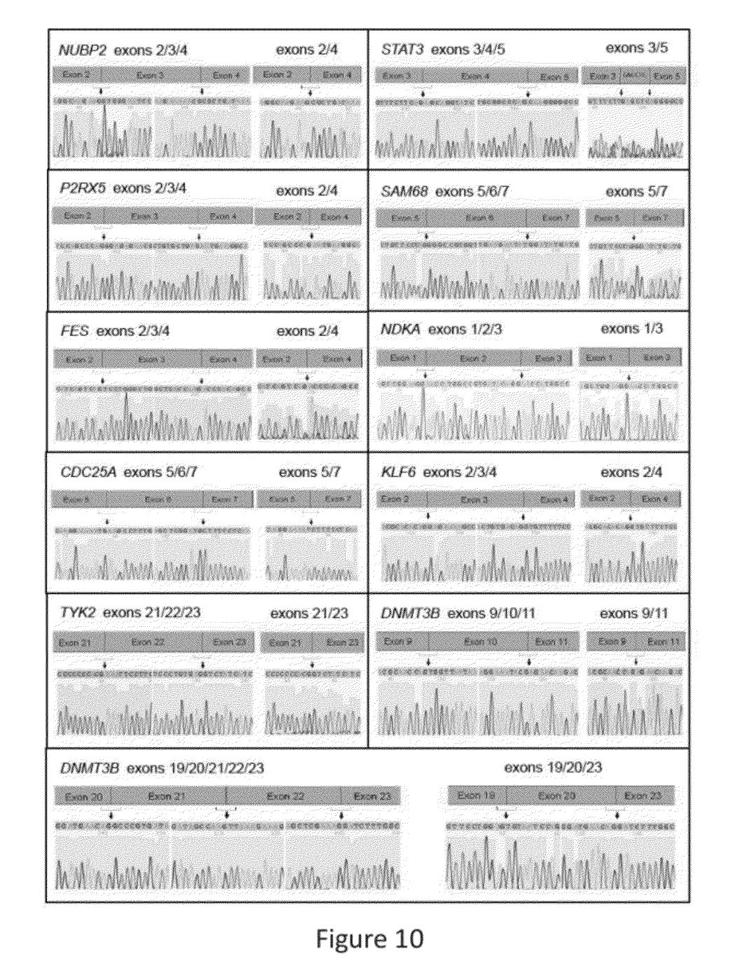

Total RNA was isolated from pluripotent stem and differentiated cells using the TRIzol method according to the manufacturer's (Invitrogen) instructions. To examine splice variant expression, cDNAs were synthesized from total RNA (2 μg) using the SuperScript II reverse transcriptase kit (Invitrogen) and random primers. PCR reactions were carried out using cDNA (1 μl) and exon-specific primers (FIG. 9) designed from information contained in alternative splicing databases such as Fast-DB (http://193.48.40.18/fastdb/), Ensembl (www.ensembl.org/index.html), Hollywood (http://hollywood.mit.edu/hollywood/) and UCSC genome browser (http://genome.ucsc.edu/). PCR products were resolved by electrophoresis on 1.5% agarose gels and visualized by ethidium bromide staining. Data were recorded using QuantityOne software (Biorad). PCR products of interest were excised, purified and directly sequenced or subcloned into pSC-A (Stratagene) and sequenced (FIG. 10). Realtime RT-PCR was performed in duplicate for each sample in an iCycler (BioRad). Reactions (25 □l) contained cDNA template (1 □l), exon-specific primers and SYBR green PCR mix (Applied Biosystems). Relative quantification was done by the □□CT method (Livak and Schmittgen, 2001).

### Peptide Antibody Development

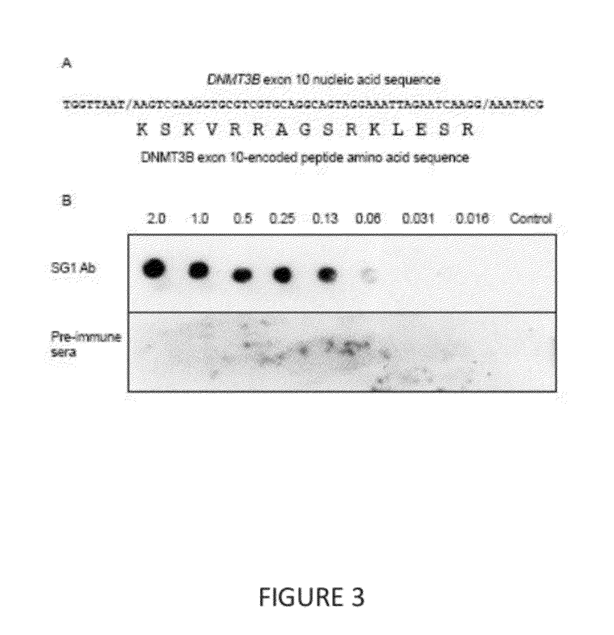

Open Biosystems (Huntsville, Ala.) synthesized the peptide, KSKVRRAGSRKLESR, encoded by DNMT3B exon 10, and produced the polyclonal antibodies. Two rabbits were injected with the above peptide conjugated to keyhole limpet hemocyanin. This study was carried out in strict accordance with the recommendations in the Guide for the Care and Use of Laboratory Animals of the National Institutes of Health. The protocol was approved by the Animal Care and Use Committee (IACUC) of Thermo Scientific, Open Biosystems (NIH (OLAW) assurance number: A3669-01; expires Mar. 31, 2012; USDA (research license) registration number: 23-R-0089; expires Jun. 6, 2011; PHS assurance number: A3669-01; expires Mar. 31, 2012). Peptide antibody specificity was determined by ELISA and the affinity purified □-DNMT3B exon 10-encoded peptide-specific rabbit polyclonal antibody was designated SG1 (FIG. 3).

### Immunofluorescence Staining of PSCs and SDCs

Cells were grown on matrigel-coated LabTek four or eight chamber slides, rinsed briefly with 1×PBS and fixed with 4% paraformaldehyde for 30 min at room temperature (RT). Samples were blocked with a solution containing 5% donkey serum and 5% Triton X100 in 1×PBS for one hour at RT, then incubated with primary antibodies at 4° C. overnight. Primary antibodies used included rabbit polyclonal α-OCT4 (1:100, Cell Signaling, catalog #2750), goat polyclonal α-OCT4 (1:100, Santa Cruz, catalog #SC-8628), mouse monoclonal α-OCT4 (POU5F1; 1:100, Sigma catalog #P0082), mouse monoclonal α-TRA-1-60 (1:500, Cell Signaling, catalog #4746), rabbit polyclonal α-DNMT3B (H-230, 1:100, Santa Cruz, catalog #20704), rabbit polyclonal α-DNMT3B (CS, 1:100, Cell Signaling, catalog #2161) and rabbit polyclonal SG1 (1:100). After overnight incubation with primary antibody, slides were washed four times with 1×PBS, incubated with secondary antibodies for one hour at RT and washed four times with 1×PBS. Secondary antibodies, purchased from R&D system, included α-mouse IgG-NL493 (catalog #NL009), α-rabbit IgG-NL493 (catalog #NL006), α-goat IgG-NL493 (catalog #NL003), α-rabbit IgG-NL557 (catalog #NL004) and α-mouse IgG-NL557 (catalog #NL007). Secondary antibodies, purchased from Invitrogen, included α-mouse IgG-Alexa fluor 488 (catalog #11017), α-mouse IgM-Alexa fluor 488 (catalog #A21042) and α-rabbbit IgG Alexa fluor 545 (catalog #A11071). All secondary antibodies were used at 1:500 dilutions. Cells were counter-stained using Hoechst/1×PBS and coverslips mounted using Vectashield mounting medium. Fluorescence images were captured on an Olympus Inverted IX81 fluorescence microscope. All images of cells are shown at 100× magnification with the exception of the two “magnification” panels in FIG. 8, which were increased in size by about 12 fold in order to allow visualization of individual cells.

### Western Blots

Pluripotent and spontaneously differentiated cells were grown on six cell matrigel-coated plates under feeder-independent conditions. Cells were rinsed twice with ice cold PBS and 0.5 to 1 ml of RIPA lysis buffer (Sigma) was added. Plates were kept at 4° C. for 5 min. Lysate was clarified by centrifugation (10,000 g, 20 min), and was used immediately or stored at −80° C. Proteins were quantified using the BCA method (Pierce). Protein (15 □g/lane) was separated by electrophoresis on a 5-15% SDS polyacrylamide gel and blotted to a nitrocellulose filter using a semi-dry blotter apparatus (Bio-Rad). Primary antibodies used for Western blots were SG1 (1:100), rabbit polyclonal α-OCT4 (1:100) and mouse monoclonal α-GAPDH (1:200, Santa Cruz, catalog #SC-47724). Secondary antibodies were horseradish peroxidase-coupled α-rabbit IgG (1:10000; Santa Cruz) or α-mouse IgG (1:10000; Santa Cruz). Secondary antibodies were detected using the ECL plus Western blotting detection system (GE Healthcare). For the dot blot assay, DNMT3B exon 10 peptide antigen was adsorbed to PVDF membrane (BioRad #162-0174) at decreasing concentrations, incubated with SG1 antibody (1:100) or pre-immune sera (1:100) followed by incubation with secondary antibody (horseradish peroxidase coupled α-rabbit IgG, 1:10000, Santa Cruz), and the blot developed using the ECL plus Western blotting detection system.

### Results

### Alternative Splice Variants of Signaling Pathway Genes are Differentially Expressed as Pluripotent Stem Cells Spontaneously Differentiate

To identify splice variants that displayed unique expression patterns in PSCs relative to SDCs, three hESC lines (H9, HES4, BG01) and one iPSC line (foreskin-1) were cultured under conditions that either maintained the pluripotent state or induced spontaneous differentiation. Genes chosen for examination included those with known or predicted splice variants that had been deposited in several alternative splicing databases (Materials and Methods) or alternatively spliced genes that had been implicated in stem cell differentiation in other organisms (Pritsker et al, 2005). An emphasis was placed on genes encoding signaling proteins because of the probability that these genes are not simply markers of ‘sternness,’ but play a functional role in maintaining the pluripotent state, and thus, may exhibit tighter regulation of splice variant expression as a function of pluripotency. Total RNA was extracted from PSCs and corresponding SDCs and subjected to semi-quantitative RT-PCR analysis using exon-specific primers. This analysis confirmed the existence of numerous alternatively spliced variants and revealed interesting changes in splicing patterns and expression ratios of splice isoforms as cells transitioned from the pluripotent to the spontaneously differentiated state (FIG. 1).

The differential splicing patterns observed in PSCs relative to SDCs fell into four general categories. In the most prevalent category, the exon-excluded splice variant was expressed at higher levels in SDCs relative to PSCs. These genes included STAT3 (signal transducer and activator of transcription 3), SAM68 (KH domain containing, RNA binding, signal transduction associated 1), KLF6 (Kruppel-like factor 6), SHCl (SHC transforming protein 1) and TBC1D3P2 (TBC1 domain family member 3, pseudogene 2) (FIG. 1). The second general category included those genes for which the exon-excluded variant was expressed at higher levels in PSCs than SDCs. Examples included FES (feline sarcoma oncogene), CDC25A (cell division cycle 25 homolog A) and TYK2 (tyrosine kinase 2). In addition to precise exon skipping, some genes selected for comparison (e.g. FES), exhibited complex changes in alternative splicing pattern including generation of a novel splice isoform that arises by utilization of a 3′ acceptor site located within the downstream exon.

For many of the splice variants, the length of the excluded exon was a multiple of three, indicating that precise exon skipping preserved the open reading frame and suggesting that the excluded exons encode important protein structural, functional or regulatory domains. For the exon-excluded splice variants, one could design a peptide encoded by sequences spanning the exon junction and use this ‘exon junction’ peptide to raise antibodies that might distinguish between proteins translated from exon-excluded vs. exon-included transcripts; however, this approach is less straightforward than generating antibodies to peptides encoded by differentially included exons.

These splice variants were found in the two remaining general categories: those genes for which the exon-included variant was expressed at higher levels in SDCs than PSCs or those for which the exon-included variant was expressed at higher levels in PSCs relative to SDCs. Among the genes analyzed, the third category was least common. In general, exon-included variants were expressed at similar or higher levels in PSCs than SDCs, indicating that the frequency of exon skipping increases after differentiation or is concomitant with loss of pluripotency. Nonetheless, for NUBP2 (nucleotide binding protein 2) the exon-included variant was expressed at higher levels in SDCs than PSCs (FIG. 1). However, overall expression of NUBP2 was low and its exon 3-included splice variant was also expressed in PSCs.

In the final category, the exon-included variant was expressed at higher levels in PSCs than SDCs. These genes included NDKA (Nucleoside diphosphate kinase A), P2RX5 (purinergic receptor P2X, ligand-gated ion channel 5) and DNMT3B (DNA cytosine-5-methyltransferase 3 beta). In the case of NDKA, the exon 2-included variant was expressed at low levels in PSCs and disappears during spontaneous differentiation. Both the exon 3-included and exon 3-excluded variants of P2RX5 were expressed at higher levels in PSCs compared to SDCs. In addition, an unknown P2RX5 splice variant that migrated above the full-length product was specifically expressed in SDCs (FIG. 1).

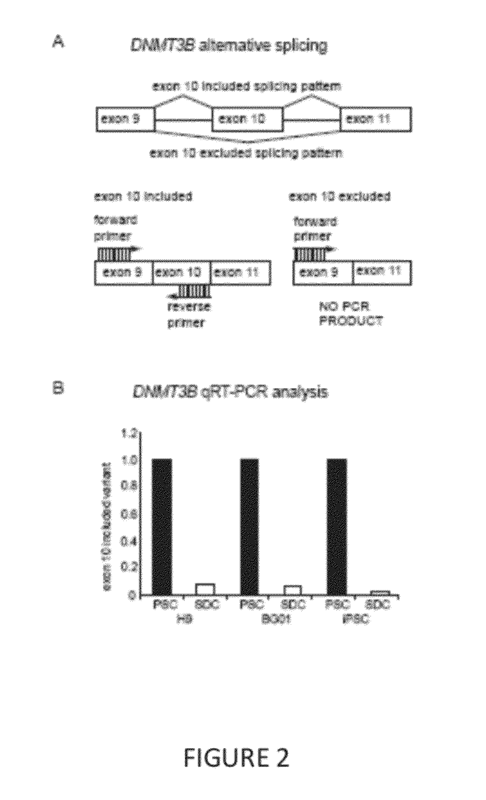

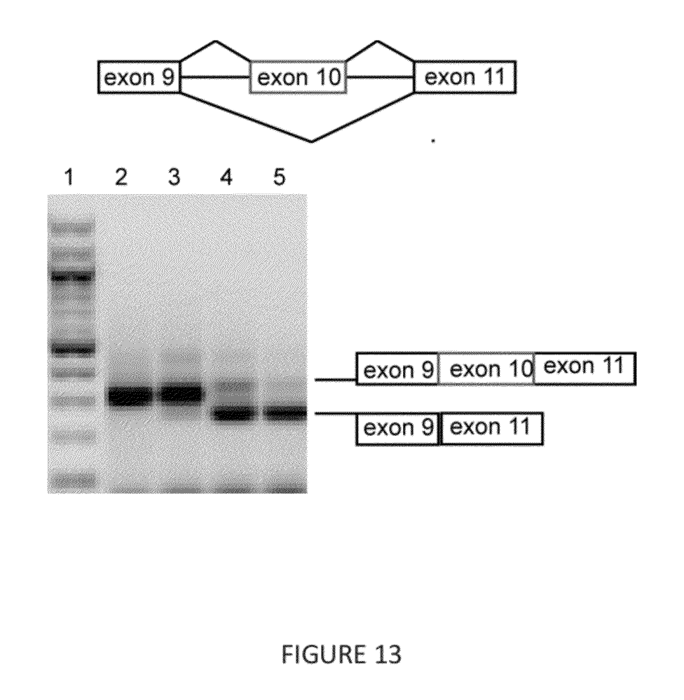

The basic structure of the DNMT3B gene from exons 9 to 11 is shown in FIG. 2A. The upper splicing pattern results in exon 10 inclusion, and is observed in pluripotent cells, while the lower splicing pattern is observed in differentiated cells, including those derived by spontaneous differentiation and those derived by directed neural differentiation. Unlike alternative splicing of the Drosophila Shaker (Sh) gene, which arises from a choice between two mutually exclusive 3′ ss, DNMT3B exon 10 is a cassette exon that is included in one context (pluripotent cells) but excluded in other (differentiated cells). Thus, the DNMT3B exon 11 3′ ss is recognized and utilized in both pluripotent and non-pluripotent cells, and DNMT3B exon 10 inclusion in PSCs does not appear to result from direct competition between the 3′ ss of exons 10 and 11 for U2AF65 binding. This is supported by intron sequences upstream of exon 10 and 11 3′ ss (FIG. 13). Intron 9/10 (upper) has all the features of a canonical, mammalian 3′ ss. The intronic AG dinucleotide located adjacent to the exon 10 3′ ss (boxed in red) is preceded by a polypyrimidine tract (PPT) of ˜30 nucleotides (underlined in green) and a branchpoint sequence (BPS, boxed in blue) that matches the mammalian BPS of YURAY (where Y is a pyrimidine and R is a purine). In addition, the PPT contains 4 repeats of the GTTTT sequence (indicated by purple line), a preferential binding site for U2AF65 that is required for U2snRNP recognition of and binding to the BPS. In contrast, intron 10/11 3′ ss (lower) deviates considerably from the consensus. In particular, the penultimate AG (boxed in yellow) is located only ˜20 nucleotides upstream of the exon 11 3′ ss, and the PPT located between the BPS and the exon 11 3′ ss is only 6 nucleotides long. Taken together this indicates that exon 10 3′ ss is ‘stronger’ than, and would ‘outcompete’, exon 11 3′ ss for U2AF65.

RT-PCR analysis of DNMT3B splice variants in PSCs relative to SDCs indicated that DNMT3B encodes at least one ideal candidate for antibody production. Two primer pairs were used to analyze expression patterns of DNMT3B splice variants. In one pair, an exon 20 forward primer was used in conjunction with an exon 23 reverse primer to examine differential expression of the DNMT3B catalytic domain encoded by exons 21 and 22. The full-length isoform was predominately expressed in PSCs, and its expression decreased, but did not disappear, during the transition from PSCs to SDCs. DNMT3B transcripts lacking exons 21 and 22 were also detected in PSCs and their expression level increased upon differentiation (FIG. 1). In contrast, the primer pair specific for exons 9 (forward) and 11 (reverse) amplified a major DNMT3B transcript containing exon 10 that was specifically expressed in PSCs and absent from SDCs. A similar finding was reported for mouse ES cells where undifferentiated cells express DNMT3B transcripts that include exon 10, while exon 10 is excluded from DNMT3B transcripts in differentiated cells (Weisenberger et al, 2004). To confirm that DNMT3B exon 10 was uniquely expressed in PSCs, realtime RT-PCR analysis was performed with an exon 9 forward primer and an exon 10 reverse primer (FIG. 2A) using RNA extracted from three pluripotent stem cell lines (H9, BG01 and iPSC) and their respective spontaneously differentiated derivates. DNMT3B exon 10 expression levels were 11-fold higher in H9 PSCs vs. H9 SDCs, 13-fold higher in BG01 PSCs vs. BG01 SDCs and 32-fold higher in iPSCs vs. iPSC SDCs (FIG. 2B).

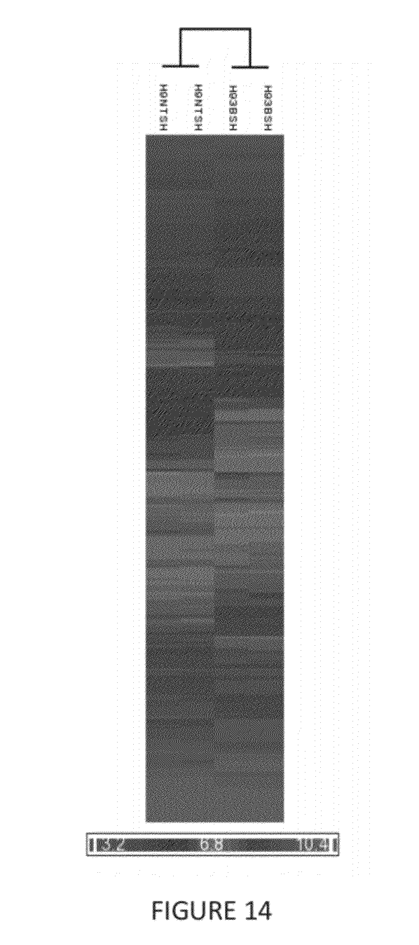

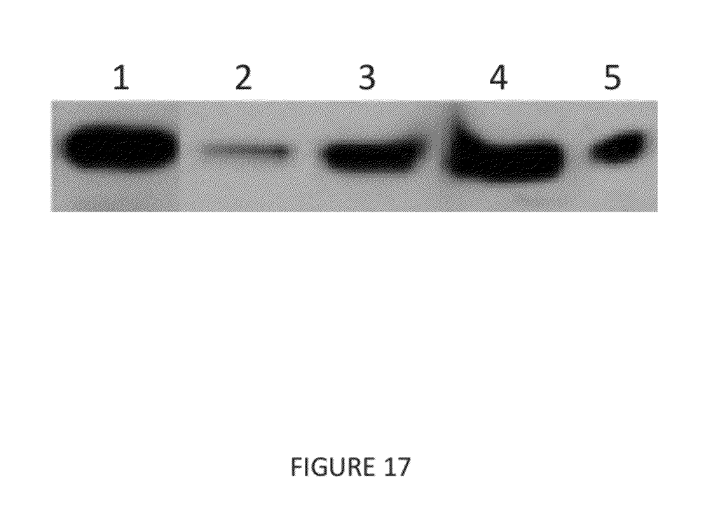

This suggests that regulation of DNMT3B exon 10 splicing involves sequences within exon 10 and/or located downstream of the exon 10 5″ splice site. Regulated alternative splicing involving intron sequences downstream of the 5″-ss has been described for a number of genes including the Src N1 exon (Chou et al., 2000) and Fas (Izquierdo et al., 2005), is often seen with cassette exons, and is generally more common when the cassette exon is relatively small. This type of regulated alternative splicing usually occurs via an ‘exon definition’ mechanism in which sequences near the downstream 5′ ss play a role in enhancing binding of U1snRNP to the 5′ ss, which is required for binding of other splicing factors that ‘bridge’ the exon and, ultimately, promote binding of U2snRNP to the upstream 3′ ss (Carlo et al., 2000). That the exon 10 5′ ss may play a role in regulating alternative splicing is also supported by the sequence located around this site, which also deviates considerably from a strong, consensus 5′ ss (FIG. 14). In particular, the GTATTT sequence (boxed in black) has three mismatches with the canonical GT(A/G)AGT sequence and the downstream sequence is enriched for pyrimidine sequences (underlined in green), including a number of potential PTB binding sites, TCTT. The presence of the downstream PPT suggests PTB may play a role in repressing DNMT3B exon 10 inclusion by interfering with U1 snRNP binding to the exon 10 5′ ss in a scenario analogous to regulated alternative splicing of Src N1 exon (Sharma et al., 2011). However, exon microarray, RNAseq and qRT-PCR analyses indicate that PTB mRNA expression is high in hESCs, and expression levels decrease (not increase) as hESCs differentiate into non-pluripotent cells. This has also been confirmed by Western blot analysis (FIG. 17 Gopalakrishna-Pillai and Iverson, unpublished results). Genome wide analysis of PTB-RNA interactions indicate PTB regulates both exon exclusion and exon inclusion, with the final outcome being determined by proximity of PTB binding sites to regulated exons and the relative strengths of PTB binding sites (Xue et al., 2009), however, exceptions to this general trend have been noted. Thus, DNMT3B exon 10 inclusion appears to be regulated either by splicing activator(s) that play a role in exon 10 5′ ss recognition that are present (or abundant) in PSCs or by splicing repressor(s) that mask the exon 10 5′ ss that are absent in PSCs but up-regulated in differentiated cells. The two hypotheses are not mutually exclusive. Complex changes in expression levels and activities of multiple splicing factors acting collectively to regulate alternative splicing event have been observed as cells differentiate, particularly along neural pathways (Gehman et al., 2012; Gehman et al., 2011; Hall et al., 2004; Rooke et al., 2003; Sharma et al., 2011).

Peptides Encoded by Alternatively Spliced Exons can be Used to Raise Antibodies that Distinguish Pluripotent Stem Cells from Early Stage Differentiated Cells.

Given the abundant, restricted expression of DNMT3B exon 10-included transcripts in PSCs relative to SDCs, this sequence was selected for peptide-specific antibody production. The peptide sequence was designed on the basis of the human DNMT3B exon 10 genomic sequence (FIG. 3A). Sequence alignment using BLAST confirmed this sequence was specific to DNMT3B exon 10 and BLASTP confirmed the peptide sequence was unique. The 15 amino acid peptide was synthesized and used as antigen for immunization of rabbits. Specificity of the SG1 antibody relative to pre-immune sera was confirmed by performing dot blot analysis against decreasing concentrations of the peptide antigen used for immunization (FIG. 3B).

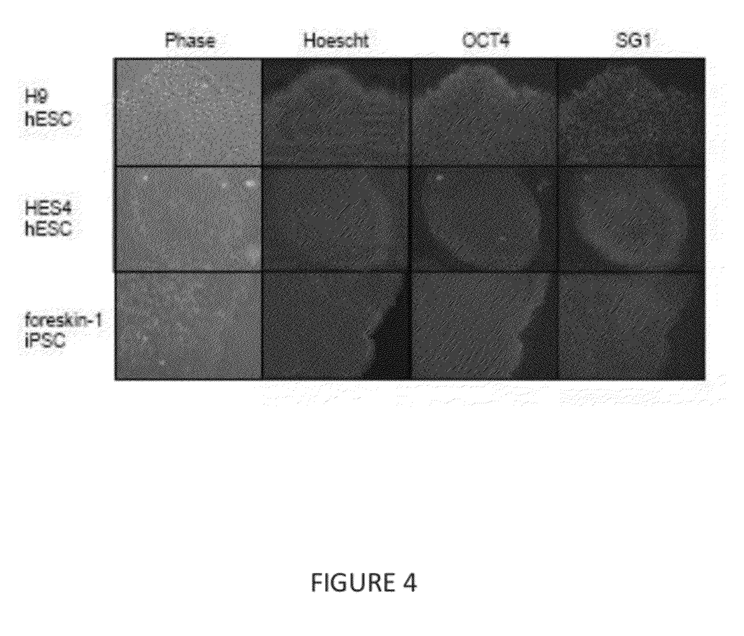

The affinity purified □-DNMT3B exon 10 encoded peptide polyclonal antibody, SG1, was first tested for its ability to detect DNMT3B expression in PSCs. Three undifferentiated pluripotent stem cell lines (H9, HES4 and iPSC) were cultured for 5-6 days and immunofluorescence staining was performed using a dual staining procedure to identify cells that stained positive with OCT4 and/or SG1 antibodies. OCT4 was chosen for comparison because it is considered a definitive marker of pluripotency. Complete overlap of OCT4 and SG1 staining was observed in both hESCs and iPSCs (FIG. 4), indicting that the SG1 antibody identifies pluripotent stem cells.

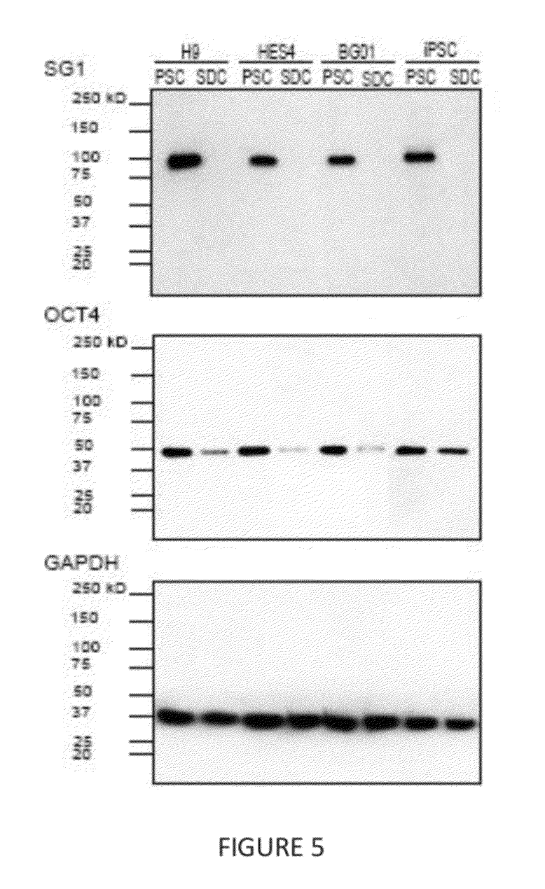

SG1 antibody was then tested for its ability to detect DNMT3B protein on Western blots of proteins extracted from four PSC lines (H9, HES4, BG01 and iPSC) and their corresponding spontaneously differentiated derivatives (14-15 days minus zbFGF). The SG1 antibody detected high-level expression of a single band of the expected MW (˜100 kD) that was present in all four PSC lines, but did not detect any protein in any of the four SDC populations (FIG. 5). In contrast, low-level expression of OCT4 protein was detected in SDCs of all three hESC lines examined. Interestingly, OCT4 expression remained fairly high in SDCs derived from the iPSC line, suggesting that the OCT4 transgene used to create this iPSC line may still be expressed even after differentiation.

To confirm the utility of the SG1 antibody at distinguishing PSCs from SDCs in mixed populations containing both pluripotent stem cells and spontaneously differentiated derivatives, two pluripotent stem cell lines (BG01 and iPSC) were examined for SG1 staining while the cells were undergoing early stages of spontaneous differentiation. Four to five day old undifferentiated PSC colonies were grown in stem cell media in the absence of zbFGF until SDCs appeared. Immunofluorescence staining of these partially differentiated colonies was performed using the SG1 antibody in comparison to the α-OCT4 and two commercially available rabbit polyclonal α-DNMT3B antibodies. Low-level OCT4 expression was detected in SDCs derived from the BG01 hESC line. Neither DNMT3B commercial antibody distinguished PSCs from SDCs (FIG. 6A). Similar results were obtained using mixed populations of PSCs and SDCs derived from the iPSC line (FIG. 6B). In marked contrast, the custom α-DNMT3B peptide antibody, SG1, was highly specific to PSCs and did not stain SDCs in mixed populations of either the BG01 hESC (FIG. 6A) or the iPSC (FIG. 6B) lines.

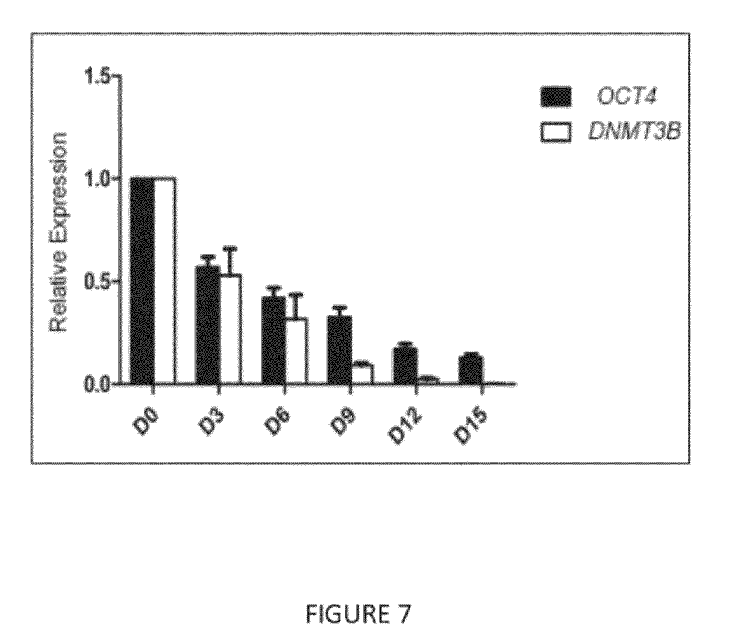

These results indicate that the SG1 antibody detects a unique DNMT3B protein isoform exhibiting an expression profile that is restricted to pluripotent stem cells. Given that expression of the DNMT3B protein isoform is down regulated faster than OCT4 protein upon spontaneous differentiation of pluripotent stem cells, transcripts containing DNMT3B exon 10 were tested to see if they also exhibit more tightly restricted expression than OCT4 transcripts in cells undergoing early stages of spontaneous differentiation. For this experiment, the time course of down regulation of DNMT3B exon 10 included transcripts was compared relative to OCT4 transcripts by quantitative RT-PCR analysis. H9 hESCs were induced to spontaneously differentiate by removal of zbFGF from the culture media. Cells were harvested at days 0, 3, 6, 9, 12 and 15, RNA extracted and qRT-PCR analysis performed using primer pairs specific for DNMT3B exon 10 (as depicted in FIG. 2A and shown in Table S1) or OCT4 (Table S1). Relative transcript expression levels were plotted as a function of number of days following induction of differentiation (FIG. 7). The time course experiment demonstrates that DNMT3B exon 10 containing transcripts also exhibit faster down regulation than OCT4 transcripts upon spontaneous differentiation. By day 6, which corresponds to early stages of spontaneous differentiation, OCT4 transcripts in SDCs are expressed at levels equivalent to 42% of that detected in PSCs, while DNMT3B exon 10 transcripts have been reduced to 32% of the original level in PSCs; by day 15 (late stage spontaneous differentiation) OCT4 transcripts in SDCs are still expressed at levels as high as about 13% the original level in PSCs, while expression levels of DNMT3B exon 10 transcripts has decreased significantly and is now expressed at levels less than 0.2% the original level observed in PSCs.

To further confirm the observed faster down regulation of the DNMT3B exon 10 encoded peptide antigen relative to other protein biomarkers of pluripotent stem cells, protein expression was re-examined in mixed cultures of PSCs and SDCs using monoclonal antibodies detecting stem cell markers, OCT4 and TRA-1-60, and compared their expression to that of the DNMT3B exon 10 encoded peptide antigen detected by the SG1 rabbit polyclonal antibody. H9 PSC colonies were grown in stem cell media in the absence of zbFGF for four to five days until SDCs appeared. Dual staining for the intracellular markers, OCT4 and DNMT3B, in mixed cultures undergoing early stage differentiation demonstrates that every cell that is detected by the SG1 antibody is also detected by the OCT4 antibody, indicating that DNMT3B exon 10 encoded peptide expression is restricted to those cells that express high-level OCT4 protein (FIG. 8A). The converse, however, is not true. A number of cells—particularly those at a distance from the main colony—exhibit high-level OCT4 expression but are not stained by the SG1 antibody, indicating that DNMT3B exon 10 encoded peptide expression is tightly restricted to PSCs, while OCT4 protein expression persists in early-stage SDCs. Similar results were obtained when using a dual staining procedure to compare DNMT3B exon 10 encoded peptide expression relative to the cell surface expressed stem cell marker, TRA-1-60 (FIG. 8B). Again, every cell detected by the SG1 antibody is also detected by the TRA-1-60 monoclonal antibody, while numerous cells—particularly those at a distance from the main colony—are stained by the TRA-1-60 antibody but not by the SG1 antibody, indicating that DNMT3B exon 10 encoded peptide expression is tightly restricted to PSCs, while TRA-1-60 protein expression persists in early-stage SDCs. Because OCT4 transcripts and OCT4 protein expression are currently considered the ‘gold standard’ for identification of pluripotent stem cells (Kellner and Kikyo, 2010), these results indicate that DNMT3Be10 and the SG1 antibody are superior reagents for distinguishing PSC's from partially differentiated derivatives and can be used to better monitor the progressive loss of ‘sternness’ as hESC's differentiate, or the progressive gain of ‘pluripotency’ during nuclear reprogramming of iPSC's.

### Discussion

Three hESC lines (H9, HES4 and BG01) and one human iPSC line (foreskin-1) were included in our study. Differential expression of alternatively spliced exons was functionally validated by RT-PCR and, in some cases, by direct sequencing. One particularly promising candidate, DNMT3B exon 10, was selected for generation of a peptide-specific polyclonal antibody, SG1. Restricted expression of DNMT3B exon 10 and the DNMT3B exon 10-encoded peptide to PSCs was confirmed by both qRT-PCR and Western blot analyses. The ability of the SG1 antibody to distinguish PSCs from SDCs was also compared to several commercially available polyclonal and monoclonal antibodies detecting stem cell proteins OCT4 and TRA-1-60. In every case, the DNMT3B exon 10-encoded peptide exhibited expression that was more restricted to PSCs. Because OCT4 transcripts and OCT4 protein expression are currently considered the ‘gold standard’ for identification of pluripotent stem cells (Kellner and Kikyo, 2010), results indicate that DNMT3B alternatively spliced exon 10 and the SG1 antibody are superior reagents for distinguishing PSCs from partially differentiated derivates and can be used to better monitor the progressive loss of ‘sternness’ as hESCs differentiate, or the progressive gain of ‘pluripotency’ during nuclear reprogramming of iPSCs.

DNMT3B is a member of the DNA methyltransferase family that was identified as a de novo methylation agent of the human genome. The human DNMT3B gene encodes as many as 40 different isoforms through alternative splicing of DNMT3B transcripts. Various DNMT3B splice isoforms are highly expressed in the human female germ line, preimplantation embryos, and embryonic stem cells, and are differentially expressed during development and tumorigenesis (Linhart et al, 2007; Beyrouthy et al, 2009; Gopalakrishnan et al, 2009). DNMT3B was identified as a commonly overexpressed marker of 59 hESC lines by microarray analysis (Adewumi et al, 2007); however, uniquely expressed splice variants are not generally detectable using conventional cDNA microarrays. DNMT3B was also suggested to be a specific marker of bona fide human pluripotent stem cells (Chan et al, 2009) based on qRT-PCR analysis that did demonstrate a high degree of specificity of expression of DNMT3B transcripts in PSCs relative to partially reprogrammed cells. However, not all DNMT3B transcripts or DNMT3B protein isoforms are unique and reliable markers of pluripotent stem cells.

DNMT3A and DNMT3B are two major de novo DNA methyltransferases. Loss of one or both results in abnormal global DNA methylation patterns; however, loss of DNMT3B (unlike DNMT3A) also results in hypomethylation of centromeric and pericentromeric satellite regions that leads to centromeric instability and mitotic defects (Hansen et al, 1999). Although the precise function of the DNMT3B exon 10-encoded peptide remains unknown, it lies between the PWWP and the ring-type zinc finger domains suggesting that it may play a role in modulating protein-protein interactions important for DNMT3B binding to H4K20me and/or targeting of DNMT3B to particular chromosomal sites (Weisenberger et al, 2004; Chen et al, 2004). A series of recent reports indicate that gene expression profiles of iPSCs and hESCs are non-identical and that some iPSCs retain an epigenetic memory of their cell type of origin that could arise from distinct global and/or gene-specific DNA methylation patterns (Chin et al, 2009; Chin et al, 2010; Deng et al, 2009; Doi et al, 2009, Guenther et al, 2010; Newman and Cooper, 2010; Kim et al, 2010). Furthermore, recent evidence indicates that the Werner Syndrome gene product, WRNp, localizes to the OCT4 promoter of human PSCs undergoing retinoic acid induced differentiation where it plays a role in de novo DNA methylation by recruiting DNMT3B to the OCT4 promoter (Smith et al, 2010). While not desiring to be bound by theory, proteins encoded by DNMT3B exon 10-containing transcripts may play a crucial role in establishing de novo DNA methylation patterns that are characteristic of the pluripotent state perhaps by regulating transcription of the pluripotency transcription factor, OCT4, and, in so doing, might affect the efficiency and/or stability of nuclear reprogrammed iPSCs. Finally, the previously noted similarities in pluripotent and cancer stem cell gene expression patterns (Clarke and Fuller, 2006) suggest that DNMT3B exon 10 may be a specific biomarker of the stem cell component of some tumors.

### Example 2

### Targeted shRNA-Mediated Knockdown of Exon 10 Containing DNMT3B Transcripts Results in a Phenotypic Defect Characterized by Chromatid Misalignment and Missegregation During Mitosis

Because the SG1 antibody has been observed to ‘paint’ the chromatids of dividing cells, indicating the DNMT3Be10 isoform binds, directly or indirectly, to DNA, shRNA-mediated targeted knockdown experiments were performed to examine the question of whether the exon 10 containing DNMT3B splice variant plays a functional role in the pluripotent stem cells in which it is expressed.

BG01 hES cells were transduced using Sigma MISSION® shRNA lentiviral transduction particle SHCLNV (TRCN0000035687) or non-target control (SHCOO2V). The viral particles were added to cultures of growing hESC's in the presence of 8 μg/μl polybrene (Sigma, St. Louis, Mo.), and incubated overnight at 37°. Stable transformants were selected for puromycin resistance (750 μg/ml) starting on day 3 after transduction. Lentiviral transduced hES cells were grown on matrigel-coated LabTek four chamber coverslips, rinsed briefly with 1×PBS, then fixed with 4% paraformaldehyde for 30 minutes at room temperature. Samples were blocked using a solution containing 5% donkey serum and 5% Triton X-100 in 1×PBS for one hour at RT, then incubated at 4° overnight with mouse monoclonal anti-β-tubulin-Cy3 antibody (1:200; Sigma catalog #C4585). Cells were washed 4× in 1×PBS and then counterstained using Hoechst in 1×PBS solution. Fluorescence images were visualized and captured with an inverted 1X81 Olympus fluorescence microscope.

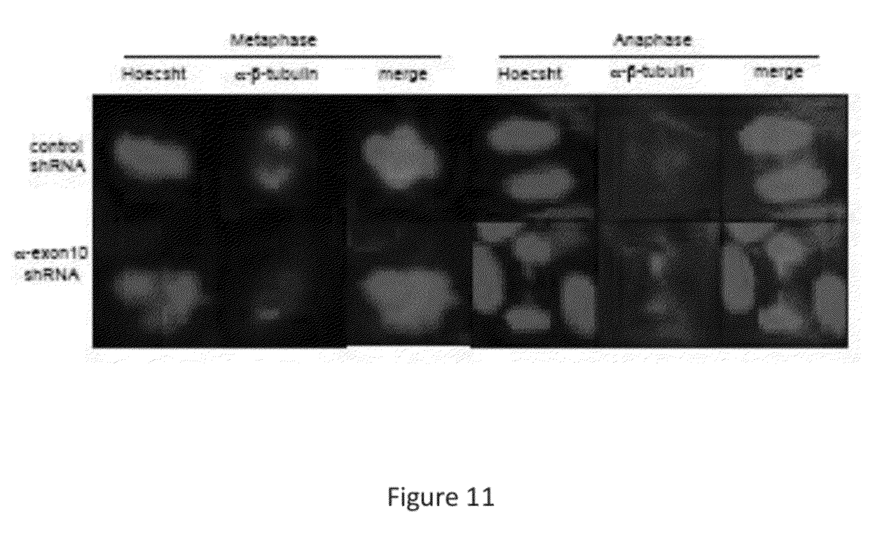

Images surprisingly reveal that, compared to controls, in which chromatids can be observed aligning at the metaphase plate, chromatids in the DNMT3B exon 10 shRNA-treated BG01 hESC's are misaligned and exhibit defects in spindle fiber formation during metaphase (FIG. 11).

Furthermore, the control shRNA cells displayed complete segregation of chromatids at anaphase, while the DNMT3B exon 10 shRNA treated cells showed evidence of chromatid missegregation during anaphase, including the presence of anaphase bridges (or lagging strands) that often give rise to aneuploidy.

This phenotypic defect is consistent with that observed for complete loss of function DNMT3B mutants, indicating that the exon 10-containing isoform plays an essential role in maintaining centromere stability during mitosis in PSC's.

This characteristic is especially relevant to the issue of aneuploidy. The mitotic defect observed in the shRNA-mediated knockdown of exon-10 containing DNMT3B transcripts can lead to aneuploidy. Spontaneous aneuploidy, particularly with respect to chromosome X, 12 and 17 trisomies, is often observed in cultured hESC's and creates numerous problems when using hESC's in regenerative medicine applications, since the aneuploid variants display characteristics of cancer cells and can be potentially tumorigenic.

### Example 3

### shRNA-Mediated Knockdown of DNMT3Be10 Alters Expression of Several Genes

Additional analysis of DNMT3Be10 shRNA-mediated knockdown reveals altered expression of canonical WNT signaling receptors encoded by the frizzled genes, FZD, that have been implicated in the stem cell self-renewal/differentiation switch (Assou et al., 2007; Cantilena et al., 2011; Katoh, 2007; Katok and Katoh, 2007; Kemp et al., 2007; Melchior et al., 2008) and the splicing factor, PRP8, that has been shown to play an essential role in 5′ splice site recognition (Grainger and Beggs, 2005) (FIG. 14).

The conserved family of secreted glycoproteins known as WNTs have been shown to regulate a wide variety of biological processes, including embryonic development, stem cell maintenance, cell fate determination, oncogenesis and suppression of tumorigenesis (Chien et al., 2009; Iglesias-Bartolome and Gutkind, 2011; Nusse, 2008). Transduction of the signal begins by WNT binding to cell-surface expressed receptors encoded by the FZD gene family, however, the array of biological activities controlled by WNT/β-catenin signaling is highly controversial. WNT has been shown to play a role in both the maintenance of stem cell self-renewal and somatic cell reprogramming as well as in controlling the exit from the stem cell state leading to lineage commitment and differentiation (Davidson et al., 2012; Mild et al., 2011; Wray and Hartmann, 2012). The divergent and opposing outcomes of WNT signaling are thought to be context and time dependent (Sokol, 2011), which may depend on FZD expression.

Results indicate that transcripts encoding both FZD7 and FZD5 receptors were DOWN-regulated upon DNMT3Be10 silencing in H9 hESCs. Over-expression of both FZD7 and FZD5 has been observed in both human and mouse ESCs (Assou et al., 2007; Kemp et al., 2007), and FZD7 has been implicated in hESC self-renewal (Melchior et al., 2008).

Interestingly, one of the genes UP-regulated by DNMT3Be10 knockdown is another WNT receptor encoded by the FZD6 gene. In contrast to FZD5 and FZD7, which are associated with self-renewal of normal embryonic stem cells, over-expression of FZD6 has been shown to be associated with differentiation and is required for neurosphere forming activity that results in highly tumorigenic stem-like cells of human neuroblastoma (Cantilena et al., 2011).

In the analysis of splice variant expression in pluripotent stem vs. spontaneously differentiated cells, a general trend emerged; pluripotent stem cells tend to exhibit more exon inclusion, while non-pluripotent cells tend to exhibit more exon exclusion (Gopalakrishna-Pillai and Iverson, 2011). A similar trend was noted previously (Pritsker et al., 2005). The fact that the essential splicing factor, PRP8 (also known as PRPF8), is also down-regulated following DNMT3Be10 KD suggests a possible explanation for this observation.

In addition to the shRNA knockdown approach used with DNMT3Be10 transcripts, lentiviral-expressed shRNA's are constructed to knockdown other DNMT3B exons, such as exons 21 and 22 encoding the methyltransferase catalytic domain; the PWWP domain, which is N-terminal to the exon 10-encoded domain and is required for targeting of DNMT3B to pericentric heterochromatin; and other DNMT's, such as DNMT3A. Because different shRNA's produce different silencing efficiencies, multiple shRNA's for each target are used to ensure maximal knockdown.

ESC's are examined for effects on gene expression and genome stability, as well as alteration of DNA methylation patterns and histone modifications. Changes in gene expression are assayed by exon microarray analysis and RNAseq analysis followed by validation of expression levels of targeted genes by qRT-PCR. Additional validation is performed by bisulfite sequencing and Chromatin Immuno-Precipitation (ChIP) analysis. The results of a typical exon microarray analysis of gene expression in H9 hESC's following α-DNMT3Be10 shRNA-mediated silencing is depicted in the heat map in FIG. 14. Unsupervised hierarchical clustering indicates excellent concordance among replicates and the inventors have discovered a relatively modest number of genes in the DNMT3Be10 shRNA group compared to the control shRNA samples. That a modest number of genes exhibited significant (p<0.05) changes in expression levels is consistent with previous studies using cells derived from ICF patients carrying mutations in the DNMT3B catalytic domain, which also reported that a relatively modest number of genes exhibited significant changes in expression levels (bot increases and decreases) in ICF cells relative to wild type cells (Jin et al., 2008). Little overlap was seen between differentially expressed genes in ICF relative to wild-type cells and differentially genes in DNMT3Be10 shRNA knocked down vs. shRNA control cells. This may reflect differences in the cellular context as Jin et. al. (2008) examined cells of lymphoblastoid lineages while examining embryonic stem cells. It may also suggest the specific targeting of DNMT3B proteins containing the exon 10-encoded domain without completely abrogating DNMT3B catalytic activity. This hypothesis can be tested using shRNAs targeting other DNMT3B domains. Of particular interest are exons 21-22 encoding the DNMT3B catalytic domain (Weisenberger et al., 2004), and exons encoding the PWWP domain, which is N-terminal to the exon 10-encoded domain and is required for targeting of DNMT3B to pericentric heterochromatin (Chen et al., 2004; Ge et al., 2004).

Typical knockdown efficiencies are between 40 and 80% decrease in expression levels within two to three days. This typically will result in an effect because SDC's appear in hESC cultures as early as two days following removal of FGF and DNMT3Be10 expression is reduced ˜50% in 3-day old SDC's. This suggests that PSC's are exquisitely sensitive to minor perturbations in DNMT3Be10 expression.

### Example 4

### Changes in DNA Methylation Patterns and Bivalent Chromatin Modifications of Differentially Expressed Genes

DNA methylation patterns of CpG islands are determined by bisulfite sequencing. Genomic DNA of shRNA-targeted and control hESC's is treated with bisulfite and used as a template for PCR amplification using gene-specific primers spanning regions of interest. PCR products are cloned and individual clones sequenced to determine the presence or absence of 5meCpG at sites of interest. Additionally, genome-wide analysis usingMethylC-seq is used (Lister et al., 2008). DNA methylation patterns are thus analyzed on cells before and after shRNA treatment. Genes are selected based upon fold-change in expression level; significance of change (p value), potential for contributing to self-renewing/differentiation switch, and the presence of a CpG island within the 5′ proximal region (within +/−1500 bp of the transcriptional start site) using the more stringent definition of a CpG island (Takai and Jones, 2002).

Epigenetic modification of DNA via methylation of CpG islands in 5′ regulatory regions has long been associated with changes in gene expression levels. Recent evidence demonstrates that histone modifications precede DNA methylation indicating that modification to the underlying histone code is a more reliable indicator of stable epigenetic changes (Rada-Iglesias and Wysocka, 2011). Thus, it is becoming increasingly clear that 5meCpG may be a surrogate marker for underlying histone modifications. Decreases in DNA methylation at CpG islands are often associated with loss of ‘repressive’ histone modifications such as H3K27me3 and gains in ‘active’ H3K4me3, but many genes showing changes in methylation status of CpG islands do not show consistent changes in bivalent chromatin modifications. This may be particularly true of DNMT3B-mediated de novo DNA methylation; only a subset of down-regulated genes in ICF patients identified by microarray analysis, validated by RT-PCR, and harboring 5′ proximal methylation of CpG islands exhibited bivalent chromatin modifications (Jin et al., 2008). The ‘processive’ nature of the DNMT3B enzyme tends to accelerate methylation at CpG rich sites (Gowher and Jeltsch, 2002), which can lead to wide spread DNA methylation that may (or may not) accurately reflect bivalent chromatin modifications that result in switching from transcriptionally ‘repressed’ to ‘active’ states. It is for these reasons that it is essential to examine histone modifications in 5′ proximal regions of the selected genes. Changes to bivalent chromatin status of 5′ regulatory regions of select genes—particularly transitions between ‘active’ H3K4me3 and ‘repressive’ H3K27me3—are assayed primarily by region-specific Chromatin Immunoprecipitation (ChIP) assays, although in some cases ChIP-seq analysis may alternatively be employed (Rada-Iglesias and Wysocka, 2011).

### Example 5

### The Effects of Targeted Silencing and Overexpression of DNMT3Be10 on hESC Self-Renewal and Differentiation

The conserved family of secreted glycoproteins known as WNTs have been shown to regulate a wide variety of biological processes, including embryonic development, stem cell maintenance, cell fate determination, oncogenesis and suppression of tumorigenesis (Chien et al., 2009; Iglesias-Bartolome and Gutkind, 2011; Nusse, 2008). Transduction of the signal begins by WNT binding to cell—surface expressed receptors encoded by the FZD gene family, however, the array of biological activities controlled by WNT/□-catenin signaling is highly controversial. WNT has been shown to play a role in both the maintenance of stem cell self-renewal and somatic cell reprogramming as well as in controlling the exit from the stem cell state leading to lineage commitment and differentiation (Davidson et al., 2012; Mild et al., 2011; Wray and Hartmann, 2012). The divergent and opposing outcomes of WNT signaling are thought to be context and time dependent (Sokol, 2011), which may depend on FZD expression.

Results indicate that transcripts encoding both FZD7 and FZD5 receptors were DOWN-regulated upon DNMT3Be10 silencing in H9 hESCs. Over-expression of both FZD7 and FZD5 has been observed in both human and mouse ESCs (Assou et al., 2007; Kemp et al., 2007), and FZD7 has been implicated in hESC self-renewal (Melchior et al., 2008). While not wishing to be bound by a particular theory, this suggests that DNMT3Be10 may participate in maintenance of the self-renewing state by promoting expression of FZD7 and FZD5. This is tested by examining the effects on gene expression of FZD7 and FZD5 KD in hESC by shRNA-mediated silencing. Since DNMT3Be10 may be acting indirectly to alter FZD expression patterns, direct silencing of FZD7 and FZD5 may have more profound effects on gene expression profiles indicative of exit from the self-renewing state and entrance into differentiated cell states. Direct silencing of FZD7 and FZD5 may also result in morphological changes indicative of spontaneous and/or neural-directed differentiation.

Interestingly, one of the genes UP-regulated by DNMT3Be10 knockdown is another WNT receptor encoded by the FZD6 gene. In contrast to FZD5 and FZD7, which are associated with self-renewal of normal embryonic stem cells, over-expression of FZD6 has been shown to be associated with differentiation and is required for neurosphere forming activity that results in highly tumorigenic stem-like cells of human neuroblastoma (Cantilena et al., 2011). The role of FZD6 in promoting differentiation or exit from the self-renewing state is determined by silencing FZD6, which may result in stabilization of the pluripotent state and by over-expressing FZD6, which may result in differentiation and, perhaps, gene expression profiles characteristic of tumorigenic stem-like neural progenitor cells (Gopalakrishna-Pillai and Iverson, 2010). Examining the balance between self-renewal promoting FZD genes vs. differentiation promoting FZD genes may help explain the long-standing conundrum regarding WNT signaling and its role in regulating the self-renewal/differentiation switch.

### Example 6

### Creation of DNMT3Be10-GFP Reporter Gene Splicing Constructs for Identifying Pluripotent Cells

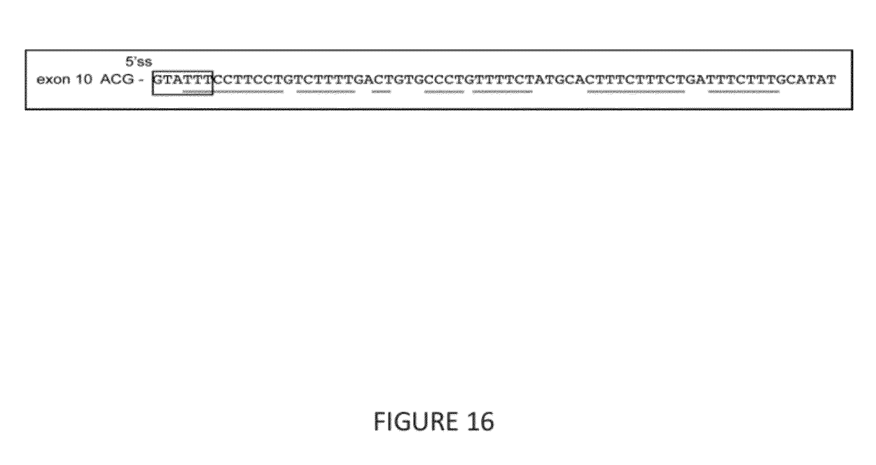

The inventors have also designed a DNMT3B splicing reporter gene construct intended to replicate the alternative splicing pattern leading to exon 10 inclusion. Upon stable transfection into hEScs (or iPSCs) this splicing reporter construct can then be used to specifically ‘mark’ bona fide pluripotent stem cells. The ‘ideal’ construct includes a promoter driving transcription that functions in both pluripotent and non-pluripotent cells, and contains the minimum DNMT3B exon/intron sequences required to recapitulate the in vivo alternative splicing pattern, which is then fused to a reporter gene that can be used to visually identify pluripotent cells in live culture and isolate pluripotent cells from non-pluripotent derivatives in mixed populations. Of particular use would be a reporter gene that can be used to isolate pluripotent cells from differentiated derivatives en masse (e.g. via flow cytometry). One preferred reporter gene is GFP. Preliminary results using a CMV promoter fused directly to GFP in a lentiviral vector indicate stable transfection (infection) of H9 hESCs cells which exhibit GFP expression in the pluripotent stem cell state, direct the hES cells to differentiate in culture into floating embryoid bodies and, eventually, neurospheres, and still observe CMV driven GFP expression (FIG. 16), demonstrating that the CMV promoter remains active following neural directed differentiation.

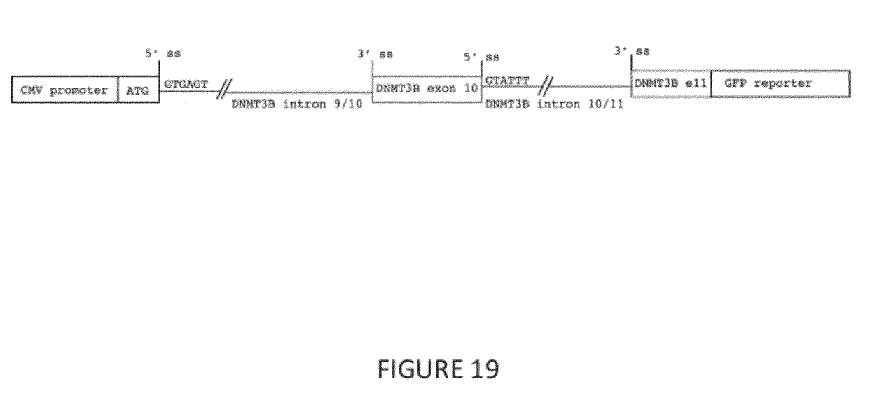

The precise design of DNMT3Be10 splicing reporter gene constructs require knowledge of the cis-sequences required to recapitulate accurate DNMT3Be10 inclusion in vitro. The identity of these sequence elements is not known at this time. Educated guesses, however, can be made based on current knowledge of alternative splicing mechanisms operating in other mammalian genes and the structure of the DNMT3B gene and flanking intron sequences (FIGS. 13 and 14). Appropriate care will be taken when designing the splicing reporter constructs to ensure that translation does initiate internally, out-of frame splicing results in translation stop signals, and—in this case—if splicing occurs between the canonical 5′ splice site and the DNMT3B exon 11 3′ splice site (i.e. if DNMT3B exon 10 is excluded from the mature mRNA) then this will result in an out-of-frame GFP reporter gene transcript incapable of translating functional GFP. The design of splicing reporter gene constructs, including splice choice vectors, that recapitulate accurate alternative splicing patterns in vivo, in vitro and in transgenic animals has been reported (Iverson et al., 1997; Mottes and Iverson, 1995). It is contemplated that additional splicing reporter gene constructs may be generated that express different colored fluorescent proteins depending on if exon 10 is included or excluded from the mature mRNA. The overall design of one possible DNMT3Be10-GFP reporter gene splicing construct is depicted in FIG. 19.

### Example 7

### The Effects of DNMT3Be10 Overexpression on iPSC Nuclear Reprogramming

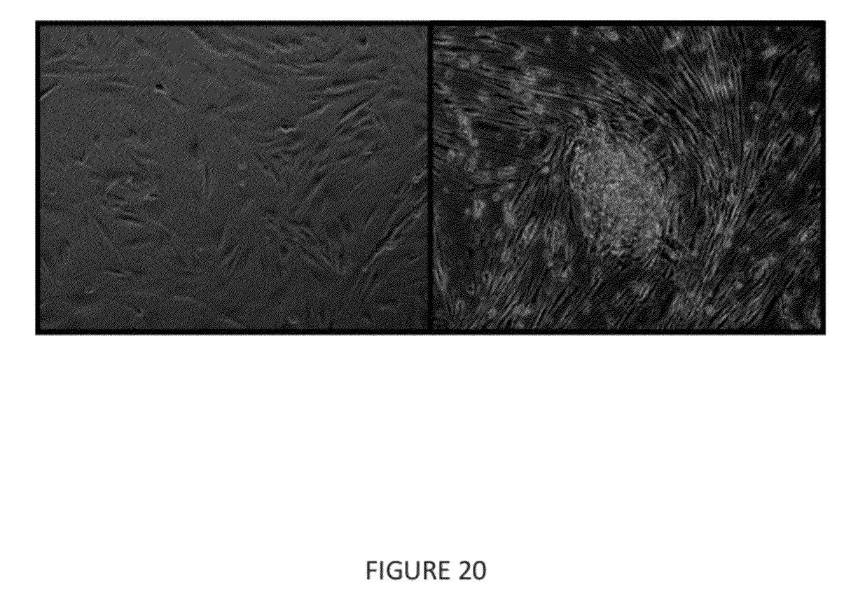

For initial iPSC generation, standard methods devised by Yamanaka and colleagues (Takahashi et al., 2007) were used in which genes encoding four transcription (Tx) factors, OCT4, KLF2, SOX2, and c-MYC, are expressed from a single lentiviral vector. An iPSC colony derived from human foreskin fibroblasts (HFF) is shown in FIG. 20. The efficiency of iPSC generation was low; of 2.0×10⁵ cells transfected, only 5 iPSC colonies were derived, and the time to iPSC generation was about 28 days. This is not an uncommon problem in nuclear reprogramming of somatic cells to iPSCs. For these experiments, HFF are co-transfected with lentiviral vectors expressing i) four Tx factors alone, ii) four Tx factors plus an additional lentiviral vector in which DNMT3Be10 expression is driven by a constitutive CMV promoter, or iii) four Tx factors plus DNMT3B□e10 (exon 10 deleted variant). Experiments are performed in triplicate. The number of iPSC colonies obtained after a fixed time period (28 days) are used to determine the frequency and efficiency of iPSC generation. Reprogrammed iPSCs are characterized by staining for expression of pluripotent stem cell markers, such as OCT4 and custom-made DNMT3Be10 peptide-specific Ab, SG1 (FIGS. 6 and 8). Additional molecular characterization of iPSC colonies includes RT-PCR analysis (FIGS. 1, 7, and 13) and Western blot analysis (FIG. 5). If DNMT3Be10 co-expression increases the % of iPSCs generated, then latter time-course experiments are carried out to determine if the increase in efficiency also results in a decrease in time, i.e. in number of days and/or number of passages required to generate bonafide pluripotent stem cells (as determined by assays described above). Finally, iPSCs generated with or without concurrent over-expression of DNMT3Be10 are used to determine if the addition of DNMT3Be10 increases the genomic stability of iPSCs as described in FIG. 11.

### Example 8

### Effect of Over-Expression of DNMT3Be10 During Nuclear Reprogramming on iPSC Fidelity

Changes in whole gene expression levels are determined for iPSCs generated in the presence of concurrent DNMT3Be10 over-expression and compared to expression profiles of iPSCs produced in absence of additional DNMT3B and/or in presence of DNMT3Be10. Both exon microarray and RNAseq analyses are used. The focus is on i) newly identified genes exhibiting highly significant (p<0.05) fold changes (both up and down) in expression levels (i.e. empirically determined genes), ii) genes identified by shRNA mediated knowckdown (KD) of DNMT3Be10 and direct shRNA mediated KD of FZD5, FZd6, FZD7, and PRP8, above, particularly if these genes play a role in the self-renewal/differentiation switch in hESCs, and iii) the 10 previously identified ‘somatic memory genes’ that exhibit persistent expression resulting from incomplete DNA methylation in iPSCs (Ohi et al., 2011). Improved fidelity of iPSCs is assessed by comparison of iPSC expression profiles to each other (+/−DNMT3Be10), by comparison with published data (Ang et al., 2011; Bar-Nur et al., 2011; Barrero and Izpisua Belmonte, 2011; Kim et al., 2010; Kim et al., 2011; Lister et al., 2011; Ohi et al., 2011; Polo et al., 2010), and by comparison to hESC expression profiles, while keeping in mind the important caveat that at least some differences in gene expression will result from underlying genetic differences in the different cell types. Differential expression initially detected by Exon microarray and RNAseq analyses can be validated by RT-PCR as shown in FIGS. 1, 7, and 13. A subset of genes identified and validated are then additional characterization of DNA methylation profiles and bivalent chromatin status is performed as described in Example 9 below.

### Example 9

### Effect of DNMT3Be10 Overexpression on Persistence of Somatic Epigenetic Events

Select genes, identified and validated according to the methods described above (including FZD 5, 6, and 7 genes) are subjected to bisulfite genomic sequencing, to determine region specific differences in DNA methylation profiles around 5′ proximal regions. Those gene exhibiting validated differential expression in reprogrammed iPSCs and differences in 5meCpG epigenetic modifications in 5′ regulatory regions of the gene in iPSCs are analyzed for underlying histone modifications. Of particular interest are the differences in ‘active’ H3K4me3 and ‘repressive’ H3K27me3 around 5′ proximal regions in incompletely reprogrammed iPSCs vs. ‘completely’ or bonafide iPSCs. Bivalent chromatin modifications are determined by ChIP analysis.

While not wishing to be bound by a particular theory, it may be concurrent over-expression of the DNMT3Be10 variant results in improved nuclear reprogramming of iPSCs. This determination is made using a combination of methods to ‘quantify’ improvement, including documenting the percentage of iPSC-like colonies produced following transfection and the passage number required to achieve iPSC-like colonies. The appearance of an iPSC-like colony following transfection of Tx factors into somatic cells does not guarantee that iPS-like cells produced are bonafide iPSCs. It is for this reason that complementary (not alternative) approaches are used (both exon microarray and RNAseq) and detailed analysis of epigenetic modifications including both 5meCpG and bivalent chromatin status. These methods are employed to identify potential candidate genes, and determine their expression levels during iPSC nuclear reprogramming. In addition, the unique DNMT3Be10-peptide specific Ab, SG1 is used, which has proven useful in identifying bonafide iPSCs. Alternative methods for iPSC generation are also being used including recently developed Sendai virus vectors expressing 4 Tx factors (Ban et al., 2011) and teratoma formation in mice is used to facilitate identification of genuine iPSCs. Additionally, vectors containing conditional promoters may be employed to determine if DNMT3Be10 over-expression is required continuously throughout the reprogramming process or if transient expression during a particular temporal window is sufficient (Yu et al., 2009). These experiments may rely on the use of fibroblasts for iPSC reprogramming, but may additionally be examined using somatic cells of all 3 lineages.

### Example 10

### Identification of Cis-Sequence Elements and Trans-Acting Factors Required for Regulated DNMT3Be10 Alternative Splicing

In vitro splicing studies are carried out using mini-gene constructs carrying DNMT3B exons 9 to 11 (and introns 9/10 and 10/11) in HeLa cell nuclear extracts. Confirmation is performed using nuclear splicing extracts derived from hESC and iPSC cultures. Mini-gene constructs carrying nested and interstitial deletions of DNMT3B intron/exon sequences, constructs carrying intron/exon insertions and point mutations are used to pinpoint the sequences necessary to recapitulate the alternative splicing pattern observed in vivo.

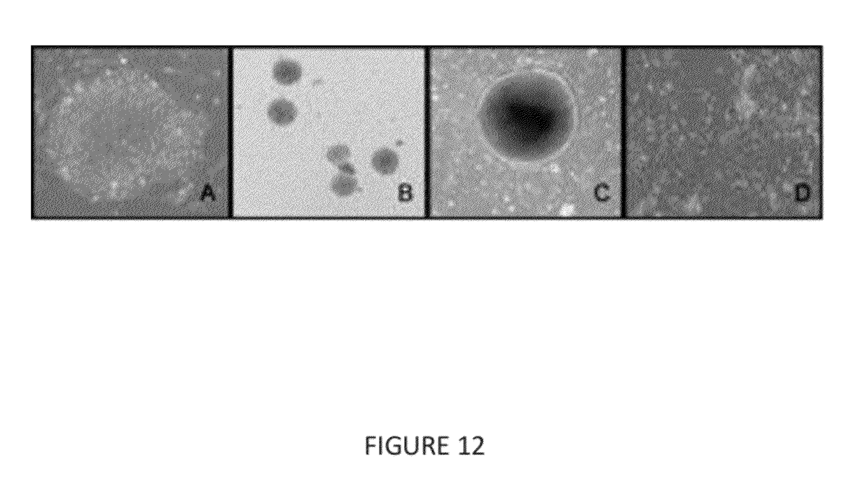

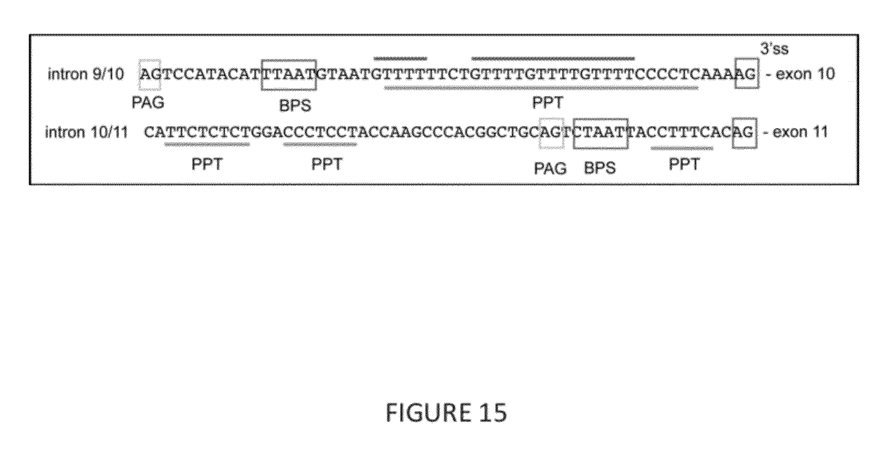

The basic structure of the DNMT3B gene from exons 9 to 11 is shown in FIG. 15. The upper splicing pattern results in exon 10 inclusion, and is observed in pluripotent cells, while the lower splicing pattern is observed in differentiated cells, including those derived by spontaneous differentiation (FIGS. 1, 7, 5, 6 and 8) and those derived by directed neural differentiation (FIGS. 12 and 13). Unlike alternative splicing of the Drosophila Sh gene which arises from a choice between two mutually exclusive 3′ splice sites (ss), DNMT3B exon 10 is a cassette exon that is included in one context (pluripotent cells) but excluded in other (differentiated cells). Thus, the DNMT3B exon 11 3′ ss is recognized and utilized in both pluripotent and non-pluripotent cells. While not wishing to be bound by a particular theory, DNMT3B exon 10 inclusion in PSCs does not appear to result from direct competition between the 3′ ss of exons 10 and 11 for U2AF65 binding. This is supported by intron sequences upstream of exon 10 and 11 3′ ss (FIG. 13). Intron 9/10 (upper) has all the features of a canonical, mammalian 3′ ss. The intronic AG dinucleotide located adjacent to the exon 10 3′ ss (boxed in red) is preceded by a polypyrimidine tract (PPT) of ˜30 nucleotides (underlined in green) and a branchpoint sequence (BPS, boxed in blue) that matches the mammalian BPS of YURAY (where Y is a pyrimidine and R is a purine). In addition, the PPT contains 4 repeats of the GTTTT sequence (indicated by purple line), a preferential binding site for U2AF65 that is required for U2snRNP recognition of and binding to the BPS. In contrast, intron 10/11 3′ ss (lower) deviates considerably from the consensus. In particular, the penultimate AG (boxed in yellow) is located only ˜20 nucleotides upstream of the exon 11 3′ ss, and the PPT located between the BPS and the exon 11 3′ ss is only 6 nucleotides long. Taken together this indicates that exon 10 3′ ss is ‘stronger’ than, and would ‘outcompete, exon 11 3’ ss for U2AF65.

This suggests that regulation of DNMT3B exon 10 splicing involves sequences within exon 10 and/or located downstream of the exon 10 5′ splice site. Regulated alternative splicing involving intron sequences downstream 5′ ss has been described for a number of genes including the Src N1 exon (Chou et al., 2000) and Fas (Izquierdo et al., 2005), is often seen with cassette exons, and is generally more common when the cassette exon is relatively small. This type of regulated alternative splicing usually occurs via an ‘exon definiton’ mechanism in which sequences near the downstream 5′ ss play a role in enhancing binding of U1snRNP to the 5′ ss, which is required for binding of other splicing factors that ‘bridge’ the exon and, ultimately, promote binding of U2snRNP to the upstream 3′ ss (Carlo et al., 2000). That the exon 10 5′ ss may play a role in regulating alternative splicing is also supported by the sequence located around this site, which also deviates considerably from a strong, consensus 5′ ss (FIG. 14). In particular, the GTATIT sequence (boxed in black) has three mismatches with the canonical GT(A/G)AGT sequence and the downstream sequence is enriched for pyrimidine sequences (underlined in green), including a number of potential PTB binding sites, TCTT. The presence of the downstream PPT suggests PTB may play a role in repressing DNMT3B exon 10 inclusion by interfering with U1 snRNP binding to the exon 10 5 ss in a scenario analogous to regulated alternative splicing of Src N1 exon (Sharma et al., 2011). However, exon microarray, RNAseq and qRT-PCR analyses indicate that PTB mRNA expression is high in hESCs, and expression levels decrease (not increase) as hESCs differentiate into non-pluripotent cells. This has also been confirmed by Western blot analysis (FIG. 17). Genome wide analysis of PTB-RNA interactions indicate that PTB regulates both exon exclusion and exon inclusion, with the final outcome being determined by proximity of PTB binding sites to regulated exons and the relative strengths of PTB binding sites (Xue et al., 2009), however, exceptions to this general trend have been noted. Thus, without intending to be bound by a particular theory, DNMT3B exon 10 inclusion is appears to be regulated either by splicing activator(s) that play a role in exon 10 5′ ss recognition that are present (or abundant) in PSCs or by splicing repressor(s) that mask the exon 10 5′ ss that are absent in PSCs but up-regulated in differentiated cells. The two hypotheses are not mutually exclusive. Complex changes in expression levels and activities of multiple splicing factors acting collectively to regulate alternative splicing event have been observed as cells differentiate, particularly along neural pathways (Gehman et al., 2012; Gehman et al., 2011; Hall et al., 2004; Rooke et al., 2003; Sharma et al., 2011). Nonetheless, sequences flanking exon 10 provide important clues for insight insight into the identity of trans-acting factors involved in regulating DNMT3B exon 10 inclusion in pluripotent cells.

After identification of cis-elements, UV crosslinking experiments, in conjunction with immunoprecipitation studies, may be employed to identify trans-acting factors that bind these sequences to regulate splice choice.

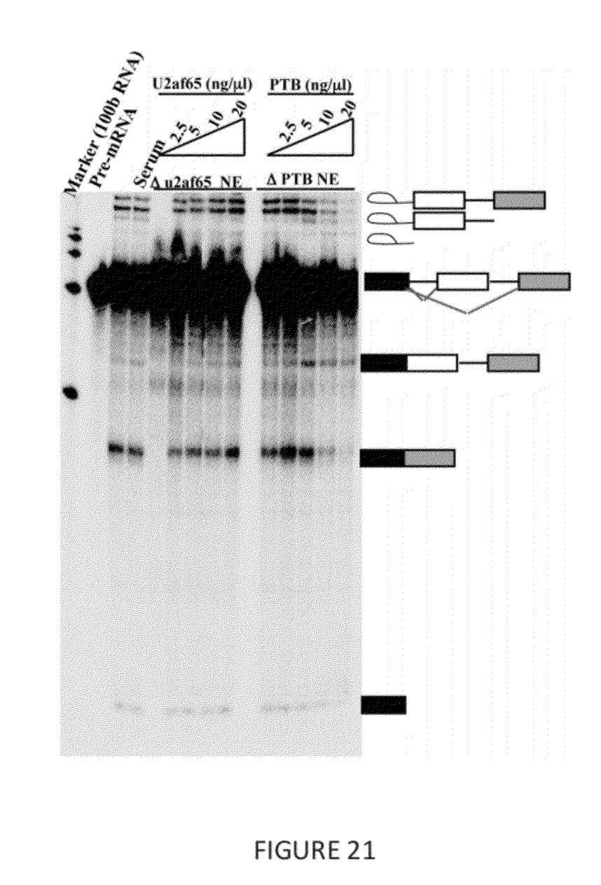

These methods have been successfully employed in studies on the regulation of alternative splicing of the Drosophila potassium ion (K+) channel gene, Shaker (Sh). Alternative splicing of Sh transcripts has been shown to account for differences in kinetic properties of Sh encoded K+ channels (Iverson and Rudy, 1990; Iverson et al., 1988). Through the use of mini-gene splicing reporter constructs, carrying nested and interstitial deletions, in transgenic animals, it was determined that only one Sh 3′ splice variant is expressed in the dorsal longitudinal muscles (DLM) of the fly (Mottes and Iverson, 1995), and this splice choice is dictated by a conserved polypyrimidine-rich sequence located in the intron upstream of the DLM-specific 3′ splice site (Iverson et al., 1997). This in vivo splicing pattern was replicated in vitro using human HeLa cell nuclear splicing extracts. UV crosslinking analysis indicated that a protein of ˜60 KD binds specifically to the cis-splicing enhancer element. Immunoprecipitation studies identified this protein as PTB (polypyrimidine tract binding protein). In mini-gene constructs, carrying both 3′ splice sites (3′ ss), depletion of U2AF65 (U2 auxiliary factor, 65 KD subunit) from nuclear extracts resulted in no splicing to either 3′ ss. The addition of U2AF65 to the reactions resulted in utilization of only the downstream 3′ ss. However, when PTB is depleted and added-back, it results in switching of 3′ ss utilization such that splicing to the downstream DLM 3′ ss is now repressed and splicing to the upstream 3′ ss is now activated (FIG. 21). This indicates that PTB competes with U2AF65. PTB acts as a repressor of one splicing event—it displaces U2AF65 from the polypyrimidine tract of the downstream intron and prevents splicing to this 3′ ss—and PTB is also an indirect activator of the other splicing event—by displacing U2AF65 from the downstream intron, U2AF65 is available to bind to the upstream intron and activate splicing to this 3′ ss.

Given the demonstrated success using these methods, one of skill in the art will recognize that no insurmountable problems exist for the use of HeLa cell nuclear extracts to recapitulate in vivo alternative splicing of the human DNMT3B gene.

The effect these splicing factors (SF) have on controlling the self-renewal/differentiation switch may be determined directly by a combination of over-expression and/or targeted shRNA-mediated silencing as described above, followed by gene expression profiling. If the identified splicing factor(s) acts to promote exon 10 inclusion in vitro, then targeted knockdown would be predicted to result in a loss of pluripotency and tip the balance toward differentiation. In contrast, if the splicing factor(s) acts to repress exon 10 inclusion, then targeted knockdown would be predicted to result in maintenance of the self-renewing pluripotent state and may act as an obstacle to differentiation. Over-expression of negative splicing factor (one that favors exon 10 exclusion) in hESCs would be expected to promote differentiation, while over-expression of a positive splicing factor (one that favors exon 10 inclusion) would be expected to promote pluripotency.

Though not intending to be bound by any particular theory, it is unlikely that any single splicing factor identified by this analysis acts solely to regulate alternative splicing of DNMT3B exon 10. Rather, it is far more likely that these splicing factors control a network of alternative splicing events that are essential for controlling the stem cell pluripotency and differentiation switch. Exon microarray and RNAseq analysis of splicing factor knockdown or over-expression (described above) are used to identify genes (transcripts) whose expressions levels and alternative splicing patterns change as a function of altering expression levels of the splicing factors. Although PTB may not be directly involved in DNMT3B exon 10 alternative splicing, given its abundance in hESCs (FIG. 17), and its well-characterized role in regulated alternative splicing, the effects of targeted knockdown and/or over-expression of PTB (and various PTB isoforms) in controlling the switch between self-renewal and differentiation in pluripotent hESCs and during nuclear reprogramming of iPSCs are determined.

Several possible candidates exist for protein or other factor(s) regulating alternative splicing of DNMT3Be10. In addition to PTB, numerous other splicing factors have been identified and shown to play a role in promoting (or suppressing) binding of U1snRNP to the 5′ ss and/or U2snRNP to the BPS. Some act directly by binding to intronic enhancer elements (e.g. U2AF65), to exonic enhancers; while some act indirectly by promoting interactions between splicing factors. A recent report indicates that not all U2snRNAs are identical and mutations in one U2snRNA gene affect alternative splicing of a network of genes (Jia et al., 2012). Thus, potential factors regulating DNMT3Be10 inclusion may be proteins, small RNAs or a combination of both; the individual role each factor plays can be assessed in in vitro splicing reactions through a series of depletion and add-back experiments as described in FIG. 12. HeLa cell nuclear extracts will be employed initially, but it is possible this cell line may lack the appropriate factors required to recapitulate accurate DNMT3Be10 splicing. Thus, we will also prepare small-scale nuclear extracts from a variety of hESC lines to identify cis-sequences and trans-acting splicing factors. Future experiments may involve the use of DNMT3Be10-GFP splicing constructs as reporter genes in high throughput screen to identify compounds that increase efficiency and/or fidelity of generation of iPSCs and/or promote or inhibit differentiation. Homogeneous PSCs isolated by GFP expression can be used to identify extracellular epitopes that may be better markers for isolating bonafide pluripotent stem cells. Expression of trans-acting splicing factors, identified in 3A, that increase DNMT3Be10 inclusion in mature transcripts can be manipulated to increase efficiency and/or fidelity of iPSCs.

While this invention has been particularly shown and described with references to preferred embodiments thereof, it will be understood by those skilled in the art that various changes in form and details may be made therein without departing from the scope of the invention encompassed by the appended claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein, including the following, are hereby incorporated by reference in their entireties. All references cited herein, whether in print, electronic, computer readable storage media or other form, are expressly incorporated by reference in their entireties, including but not limited to, abstracts, articles, journals, publications, texts, treatises, internet web sites, databases, patents, and patent publications.

### REFERENCES

Adewumi, O., Aflatoonian, B., Ahrlund-Richter, L., Amit, M., Andrews, P. W., Beighton, G., Bello, P. A., Benvenisty, N., Berry, L. S., Bevan, S., et al. (2007). Characterization of human embryonic stem cell lines by the International Stem Cell Initiative. Nat. Biotechnol 25, 803-816.
Ang, Y. S., Gaspar-Maia, A., Lemischka, I. R., and Bernstein, E. (2011). Stem cells and reprogramming: breaking the epigenetic barrier? Trends Pharmacol Sci 32, 394-401.
Assou, S., Le Carrour, T., Tondeur, S., Strom, S., Gabelle, A., Marty, S., Nadal, L., Pantesco, V., Reme, T., Hugnot, J. P., et al. (2007). A meta-analysis of human embryonic stem cells transcriptome integrated into a web-based expression atlas. Stem Cells 25, 961-973.
Athanasiadou, R., de Sousa, D., Myant, K., Merusi, C., Stancheva, I., and Bird, A. (2010). Targeting of de novo DNA methylation throughout the Oct-4 gene regulatory region in differentiating embryonic stem cells. PLoS One 5, e9937.
Bachman, K. E., Rountree, M. R., and Baylin, S. B. (2001). Dnmt3a and Dnmt3b are transcriptional repressors that exhibit unique localization properties to heterochromatin. J Biol Chem 276, 32282-32287.
Ban, H., Nishishita, N., Fusaki, N., Tabata, T., Saeki, K., Shikamura, M., Takada, N., Inoue, M., Hasegawa, M., Kawamata, S., et al. (2011). Efficient generation of transgene-free human induced pluripotent stem cells (iPSCs) by temperature-sensitive Sendai virus vectors. Proc Natl Acad Sci USA 108, 14234-14239.
Bar-Nur, O., Russ, H. A., Efrat, S., and Benvenisty, N. (2011). Epigenetic memory and preferential lineage-specific differentiation in induced pluripotent stem cells derived from human pancreatic islet beta cells. Cell Stem Cell 9, 17-23.
Barrero, M. J., and Izpisua Belmonte, J. C. (2011). iPS cells forgive but do not forget. Nat Cell Biol 13, 523-525.
Beaulieu, N., Morin, S., Chute, I. C., Robert, M. F., Nguyen, H., and MacLeod, A. R. (2002). An essential role for DNA methyltransferase DNMT3B in cancer cell survival. J Biol Chem 277, 28176-28181.
Beyrouthy, M. J., Garner, K. M., Hever, M. P., Freemantle, S. J., Eastman, A., Dmitrovsky, E., and Spinella, M. J. (2009). High DNA methyltransferase 3B expression mediates 5-aza-deoxycytidine hypersensitivity in testicular germ cell tumors. Cancer Res 69, 9360-9366.
Blasco, M. A., Serrano, M., and Fernandez-Capetillo, O. (2011). Genomic instability in iPS: time for a break. EMBO J 30, 991-993.
Cantilena, S., Pastorino, F., Pezzolo, A., Chayka, O., Pistoia, V., Ponzoni, M., and Sala, A. (2011). Frizzled receptor 6 marks rare, highly tumourigenic stem-like cells in mouse and human neuroblastomas. Oncotarget 2, 976-983.
Carlo, T., Sierra, R., and Berget, S. M. (2000). A 5′ splice site-proximal enhancer binds SF1 and activates exon bridging of a microexon. Mol Cell Biol 20, 3988-3995.
Challen, G. A., Sun, D., Jeong, M., Luo, M., Jelinek, J., Berg, J. S., Bock, C., Vasanthakumar, A., Gu, H., Xi, Y., et al. (2012). Dnmt3a is essential for hematopoietic stem cell differentiation. Nat Genet 44, 23-31.
Chan, E. M., Ratanasirintrawoot, S., Park, I. H., Manos, P. D., Loh, Y. H., Huo, H., Miller, J. D., Hartung, O., Rho, J., Ince, T. A., et al. (2009). Live cell imaging distinguishes bona fide human iPS cells from partially reprogrammed cells. Nat Biotechnol 27, 1033-1037.
Chen, L., Huang, S., Lee, L., Davalos, A., Schiestl, R. H., Campisi, J., and Oshima, J. (2003a). WRN, the protein deficient in Werner syndrome, plays a critical structural role in optimizing DNA repair. Aging Cell 2, 191-199.
Chen, T., Tsujimoto, N., and Li, E. (2004). The PWWP domain of Dnmt3a and Dnmt3b is required for directing DNA methylation to the major satellite repeats at pericentric heterochromatin. Mol Cell Biol 24, 9048-9058.
Chen, T., Ueda, Y., Dodge, J. E., Wang, Z., and Li, E. (2003b). Establishment and maintenance of genomic methylation patterns in mouse embryonic stem cells by Dnmt3a and Dnmt3b. Mol Cell Biol 23, 5594-5605.
Chien, A. J., Conrad, W. H., and Moon, R. T. (2009). A Wnt survival guide: from flies to human disease. J Invest Dermatol 129, 1614-1627.
Chin, M. H., et al., Induced pluripotent stem cells and embryonic stem cells are distinguished by gene expression signatures. Cell Stem Cell, 2009. 5(1): p. 111-23.
Chin, M. H., et al., Molecular analyses of human induced pluripotent stem cells and embryonic stem cells. Cell Stem Cell, 2010. 7(2): p. 263-9.
Chou, M. Y., Underwood, J. G., Nikolic, J., Luu, M. H., and Black, D. L. (2000). Multisite RNA binding and release of polypyrimidine tract binding protein during the regulation of c-src neural-specific splicing. Mol Cell 5, 949-957.
Clarke, M. F., and Fuller, M. (2006). Stem cells and cancer: two faces of eve. Cell 124, 1111-1115.
Davidson, K. C., Adams, A. M., Goodson, J. M., McDonald, C. E., Potter, J. C., Berndt, J. D., Biechele, T. L., Taylor, R. J., and Moon, R. T. (2012). Wnt/beta-catenin signaling promotes differentiation, not self-renewal, of human embryonic stem cells and is repressed by Oct4. Proc Natl Acad Sci USA 109, 4485-4490.
Deng, W., and Xu, Y. (2009). Genome integrity: linking pluripotency and tumorgenicity. Trends Genet 25, 425-427.
Deng, J., et al., Targeted bisulfite sequencing reveals changes in DNA methylation associated with nuclear reprogramming. Nat Biotechnol, 2009. 27(4): p. 353-60.
Doi, A., et al., Differential methylation of tissue- and cancer-specific CpG island shores distinguishes human induced pluripotent stem cells, embryonic stem cells and fibroblasts. Nat Genet, 2009. 41(12): p. 1350-3.
Ehrlich, M., Jackson, K., and Weemaes, C. (2006). Immunodeficiency, centromeric region instability, facial anomalies syndrome (ICF). Orphanet J Rare Dis 1, 2.
Gabut, M., Samavarchi-Tehrani, P., Wang, X., Slobodeniuc, V., O'Hanlon, D., Sung, H. K., Alvarez, M., Talukder, S., Pan, Q., Mazzoni, E. O., et al. (2011). An alternative splicing switch regulates embryonic stem cell pluripotency and reprogramming. Cell 147, 132-146.
Ge, Y. Z., Pu, M. T., Gowher, H., Wu, H. P., Ding, J. P., Jeltsch, A., and Xu, G. L. (2004). Chromatin targeting of de novo DNA methyltransferases by the PWWP domain. J Biol Chem 279, 25447-25454.
Gellman, L. T., Meera, P., Stoilov, P., Shiue, L., O'Brien, J. E., Meisler, M. H., Ares, M., Jr., Otis, T. S., and Black, D. L. (2012). The splicing regulator Rbfox2 is required for both cerebellar development and mature motor function. Genes Dev 26, 445-460.
Gehman, L. T., Stoilov, P., Maguire, J., Damianov, A., Lin, C. H., Shiue, L., Ares, M., Jr., Mody, I., and Black, D. L. (2011). The splicing regulator Rbfox1 (A2BP1) controls neuronal excitation in the mammalian brain. Nat Genet 43, 706-711.
Gopalakrishna-Pillai, S., and Iverson, L. E. (2010). Astrocytes derived from trisomic human embryonic stem cells express markers of astrocytic cancer cells and premalignant stem-like progenitors. BMC Med Genomics 3, 12.
Gopalakrishna-Pillai, S., and Iverson, L. E. (2011). A DNMT3B alternatively spliced exon and encoded peptide are novel biomarkers of human pluripotent stem cells. PLoS One 6, e20663.
Gopalakrishnan, S., Sullivan, B. A., Trazzi, S., Della Valle, G., and Robertson, K. D. (2009a). DNMT3B interacts with constitutive centromere protein CENP-C to modulate DNA methylation and the histone code at centromeric regions. Hum Mol Genet 18, 3178-3193.
Gopalakrishnan, S., Van Emburgh, B. O., Shan, J., Su, Z., Fields, C. R., Vieweg, J., Hamazaki, T., Schwartz, P. H., Terada, N., and Robertson, K. D. (2009b). A novel DNMT3B splice variant expressed in tumor and pluripotent cells modulates genomic DNA methylation patterns and displays altered DNA binding. Mol Cancer Res 7, 1622-1634.
Gowher, H., and Jeltsch, A. (2002). Molecular enzymology of the catalytic domains of the Dnmt3a and Dnmt3b DNA methyltransferases. J Biol Chem 277, 20409-20414.
Grainger, R. J., and Beggs, J. D. (2005). Prp8 protein: at the heart of the spliceosome. RNA 11, 533-557.
Guenther, M. G., et al., Chromatin structure and gene expression programs of human embryonic and induced pluripotent stem cells. Cell Stem Cell, 2010. 7(2): p. 249-57.
Hall, M. P., Huang, S., and Black, D. L. (2004). Differentiation-induced colocalization of the KH-type splicing regulatory protein with polypyrimidine tract binding protein and the c-src pre-mRNA. Mol Biol Cell 15, 774-786.
Hansen, R. S., et al., The DNMT3B DNA methyltransferase gene is mutated in the ICF immunodeficiency syndrome. Proc Natl Acad Sci USA, 1999. 96(25): p. 14412-7.
Hervouet, E., Vallette, F. M., and Cartron, P. F. (2009). Dnmt3/transcription factor interactions as crucial players in targeted DNA methylation. Epigenetics 4, 487-499.
Hu, G., Huang, K., Yu, J., Gopalakrishna-Pillai, S., Kong, J., Xu, H., Liu, Z., Zhang, K., Xu, J., Luo, Y., et al. (2012). Identification of miRNA signatures during the differentiation of hESCs into retinal pigment epithelial cells. PLoS One submitted Jan. 23, 2012; under revision.
Huntriss, J., Hinkins, M., Oliver, B., Harris, S. E., Beazley, J. C., Rutherford, A. J., Gosden, R. G., Lanzendorf, S. E., and Picton, H. M. (2004). Expression of mRNAs for DNA methyltransferases and methyl-CpG-binding proteins in the human female germ line, preimplantation embryos, and embryonic stem cells. Mol Reprod Dev 67, 323-336.
Iglesias-Bartolome, R., and Gutkind, J. S. (2011). Signaling circuitries controlling stem cell fate: to be or not to be. Curr Opin Cell Biol 23, 716-723.
Iverson, L. E., Mottes, J. R., Yeager, S. A., and Germeraad, S. E. (1997). Tissue-specific alternative splicing of Shaker potassium channel transcripts results from distinct modes of regulating 3′ splice choice. J Neurobiol 32, 457-468.
Iverson, L. E., and Rudy, B. (1990). The role of the divergent amino and carboxyl domains on the inactivation properties of potassium channels derived from the Shaker gene of Drosophila. J Neurosci 10, 2903-2916.
Iverson, L. E., Tanouye, M. A., Lester, H. A., Davidson, N., and Rudy, B. (1988). A-type potassium channels expressed from Shaker locus cDNA. Proc Natl Acad Sci USA 85, 5723-5727.
Izquierdo, J. M., Majos, N., Bonnal, S., Martinez, C., Castelo, R., Guigo, R., Bilbao, D., and Valcarcel, J. (2005). Regulation of Fas alternative splicing by antagonistic effects of TIA-1 and PTB on exon definition. Mol Cell 19, 475-484.
Jia, Y., Mu, J. C., and Ackerman, S. L. (2012). Mutation of a U2 snRNA gene causes global disruption of alternative splicing and neurodegeneration. Cell 148, 296-308.
Jin, B., Tao, Q., Peng, J., Soo, H. M., Wu, W., Ying, J., Fields, C. R., Delmas, A. L., Liu, X., Qiu, J., et al. (2008). DNA methyltransferase 3B (DNMT3B) mutations in ICF syndrome lead to altered epigenetic modifications and aberrant expression of genes regulating development, neurogenesis and immune function. Hum Mol Genet 17, 690-709.
Katoh, M. (2007). Comparative integromics on FZD7 orthologs: conserved binding sites for PU.1, SP1, CCAAT-box and TCF/LEF/SOX transcription factors within 5′-promoter region of mammalian FZD7 orthologs. Int J Mol Med 19, 529-533.
Katoh, Y., and Katoh, M. (2007). Conserved POU-binding site linked to SP1-binding site within FZD5 promoter: Transcriptional mechanisms of FZD5 in undifferentiated human ES cells, fetal liver/spleen, adult colon, pancreatic islet, and diffuse-type gastric cancer. Int J Oncol 30, 751-755.
Kellner, S. and N. Kikyo, Transcriptional regulation of the Oct4 gene, a master gene for pluripotency. Histol Histopathol, 2010. 25(3): p. 405-12.
Kemp, C. R., Willems, E., Wawrzak, D., Hendrickx, M., Agbor Agbor, T., and Leyns, L. (2007). Expression of Frizzled5, Frizzled7, and Frizzled10 during early mouse development and interactions with canonical Wnt signaling. Dev Dyn 236, 2011-2019.
Kim, K., Doi, A., Wen, B., Ng, K., Zhao, R., Cahan, P., Kim, J., Aryee, M. J., Ji, H., Ehrlich, L. I., et al. (2010). Epigenetic memory in induced pluripotent stem cells. Nature 467, 285-290.
Kim, K., Zhao, R., Doi, A., Ng, K., Untemaehrer, J., Cahan, P., Huo, H., Loh, Y. H., Aryee, M. J., Lensch, M. W., et al. (2011). Donor cell type can influence the epigenome and differentiation potential of human induced pluripotent stem cells. Nat Biotechnol 29, 1117-1119.
Linhart, H. G., et al., Dnmt3b promotes tumorigenesis in vivo by gene-specific de novo methylation and transcriptional silencing. Genes Dev, 2007. 21(23): p. 3110-22.
Lister, R., O'Malley, R. C., Tonti-Filippini, J., Gregory, B. D., Berry, C. C., Millar, A. H., and Ecker, J. R. (2008). Highly integrated single-base resolution maps of the epigenome in Arabidopsis. Cell 133, 523-536.
Lister, R., Pelizzola, M., Kida, Y. S., Hawkins, R. D., Nery, J. R., Hon, G., Antosiewicz-Bourget, J., O'Malley, R., Castanon, R., Klugman, S., et al. (2011). Hotspots of aberrant epigenomic reprogramming in human induced pluripotent stem cells. Nature 471, 68-73.
Livak, K. J. and T. D. Schmittgen, Analysis of relative gene expression data using real-time quantitative PCR and the 2(-Delta Delta C(T)) Method. Methods, 2001. 25(4): p. 402-8.
Ludwig, T. E., et al., Feeder-independent culture of human embryonic stem cells. Nat Methods, 2006. 3(8): p. 637-46.
Martins-Taylor, K., Schroeder, D. I., Lasalle, J. M., Lalande, M., and Xu, R. H. (2012). Role of DNMT3B in the regulation of early neural and neural crest specifiers. Epigenetics 7.
Matarazzo, M. R., De Bonis, M. L., Vacca, M., Della Ragione, F., and DEsposito, M. (2009). Lessons from two human chromatin diseases, ICF syndrome and Rett syndrome. Int J Biochem Cell Biol 41, 117-126.
Melchior, K., Weiss, J., Zaehres, H., Kim, Y. M., Lutzko, C., Roosta, N., Hescheler, J., and Muschen, M. (2008). The WNT receptor FZD7 contributes to self-renewal signaling of human embryonic stem cells. Biol Chem 389, 897-903.
Miki, T., Yasuda, S. Y., and Kahn, M. (2011). Wnt/beta-catenin signaling in embryonic stem cell self-renewal and somatic cell reprogramming. Stem Cell Rev 7, 836-846.
Mottes, J. R., and Iverson, L. E. (1995). Tissue-specific alternative splicing of hybrid Shaker/lacZ genes correlates with kinetic differences in Shaker K+ currents in vivo. Neuron 14, 613-623.
Newman, A. M. and J. B. Cooper, Lab-specific gene expression signatures in pluripotent stem cells. Cell Stem Cell, 2010. 7(2): p. 258-62.
Nicholas, C. R. and A. R. Kriegstein, Regenerative medicine: Cell reprogramming gets direct. Nature, 2010. 463(7284): p. 1031-2.
Nusse, R. (2008). Wnt signaling and stem cell control. Cell Res 18, 523-527.
Odorico, J. S., D. S. Kaufman, and J. A. Thomson, Multilineage differentiation from human embryonic stem cell lines. Stem Cells, 2001. 19(3): p. 193-204.
Ohi, Y., Qin, H., Hong, C., Blouin, L., Polo, J. M., Guo, T., Qi, Z., Downey, S. L., Manos, P. D., Rossi, D. J., et al. (2011). Incomplete DNA methylation underlies a transcriptional memory of somatic cells in human iPS cells. Nat Cell Biol 13, 541-549.
Okano, M., Bell, D. W., Haber, D. A., and Li, E. (1999). DNA methyltransferases Dnmt3a and Dnmt3b are essential for de novo methylation and mammalian development. Cell 99, 247-257.
Orengo, J. P. and T. A. Cooper, Alternative splicing in disease. Adv Exp Med Biol, 2007. 623: p. 212-23.
Ostler, K. R., Davis, E. M., Payne, S. L., Gosalia, B. B., Exposito-Cespedes, J., Le Beau, M. M., and Godley, L. A. (2007). Cancer cells express aberrant DNMT3B transcripts encoding truncated proteins. Oncogene 26, 5553-5563.
Polo, J. M., Liu, S., Figueroa, M. E., Kulalert, W., Eminli, S., Tan, K. Y., Apostolou, E., Stadtfeld, M., Li, Y., Shioda, T., et al. (2010). Cell type of origin influences the molecular and functional properties of mouse induced pluripotent stem cells. Nat Biotechnol 28, 848-855.
Pritsker, M., Doniger, T. T., Kramer, L. C., Westcot, S. E., and Lemischka, I. R. (2005). Diversification of stem cell molecular repertoire by alternative splicing. Proc Natl Acad Sci USA 102, 14290-14295.
Rada-Iglesias, A., and Wysocka, J. (2011). Epigenomics of human embryonic stem cells and induced pluripotent stem cells: insights into pluripotency and implications for disease. Genome Med 3, 36.
Rooke, N., Markovtsov, V., Cagavi, E., and Black, D. L. (2003). Roles for SR proteins and hnRNP A1 in the regulation of c-src exon N1. Mol Cell Biol 23, 1874-1884.
Salomonis, N., Schlieve, C. R., Pereira, L., Wahlquist, C., Colas, A., Zambon, A. C., Vranizan, K., Spindler, M. J., Pico, A. R., Cline, M. S., et al. (2010). Alternative splicing regulates mouse embryonic stem cell pluripotency and differentiation. Proc Natl Acad Sci USA 107, 10514-10519.
Sharma, S., Maris, C., Allain, F. H., and Black, D. L. (2011). U1 snRNA directly interacts with polypyrimidine tract-binding protein during splicing repression. Mol Cell 41, 579-588.
Smith, J. A., Ndoye, A. M., Geary, K., Lisanti, M. P., Igoucheva, O., and Daniel, R. (2010). A role for the Werner syndrome protein in epigenetic inactivation of the pluripotency factor Oct4. Aging Cell 9, 580-591.
Sokol, S. Y. (2011). Maintaining embryonic stem cell pluripotency with Wnt signaling. Development 138, 4341-4350.
Stewart, S. A., Dykxhoorn, D. M., Palliser, D., Mizuno, H., Yu, E. Y., An, D. S., Sabatini, D. M., Chen, I. S., Hahn, W. C., Sharp, P. A., et al. (2003). Lentivirus-delivered stable gene silencing by RNAi in primary cells. RNA 9, 493-501.
Sultan, M., Schulz, M. H., Richard, H., Magen, A., Klingenhoff, A., Scherf, M., Seifert, M., Borodina, T., Soldatov, A., Parkhomchuk, D., et al. (2008). A global view of gene activity and alternative splicing by deep sequencing of the human transcriptome. Science 321, 956-960.
Taapken, S. M., Nisler, B. S., Newton, M. A., Sampsell-Barron, T. L., Leonhard, K. A., McIntire, E. M., and Montgomery, K. D. (2011). Karotypic abnormalities in human induced pluripotent stem cells and embryonic stem cells. Nat Biotechnol 29, 313-314.
Takahashi, K., Tanabe, K., Ohnuki, M., Narita, M., Ichisaka, T., Tomoda, K., and Yamanaka, S. (2007). Induction of pluripotent stem cells from adult human fibroblasts by defined factors. Cell 131, 861-872.
Takai, D., and Jones, P. A. (2002). Comprehensive analysis of CpG islands in human chromosomes 21 and 22. Proc Natl Acad Sci USA 99, 3740-3745.
Trapnell, C., Williams, B. A., Pertea, G., Mortazavi, A., Kwan, G., van Baren, M. J., Salzberg, S. L., Wold, B. J., and Pachter, L. (2010). Transcript assembly and quantification by RNA-Seq reveals unannotated transcripts and isoform switching during cell differentiation. Nat Biotechnol 28, 511-515.
Turaga, R. V., Massip, L., Chavez, A., Johnson, F. B., and Lebel, M. (2007). Werner syndrome protein prevents DNA breaks upon chromatin structure alteration. Aging Cell 6, 471-481.
Weisenberger, D. J., Velicescu, M., Cheng, J. C., Gonzales, F. A., Liang, G., and Jones, P. A. (2004). Role of the DNA methyltransferase variant DNMT3b3 in DNA methylation. Mol Cancer Res 2, 62-72.
Wray, J., and Hartmann, C. (2012). WNTing embryonic stem cells. Trends Cell Biol 22, 159-168.
Xue, Y., Zhou, Y., Wu, T., Zhu, T., Ji, X., Kwon, Y. S., Zhang, C., Yeo, G., Black, D. L., Sun, H., et al. (2009). Genome-wide analysis of PTB-RNA interactions reveals a strategy used by the general splicing repressor to modulate exon inclusion or skipping. Mol Cell 36, 996-1006.
Yeo, G. W., et al., Alternative splicing events identified in human embryonic stem cells and neural progenitors. PLoS Comput Biol, 2007. 3(10): p. 1951-67.
Yu, J., et al., Induced pluripotent stem cell lines derived from human somatic cells. Science, 2007. 318(5858): p. 1917-20.
Yu, J., Hu, K., Smuga-Otto, K., Tian, S., Stewart, R., Slukvin, II, and Thomson, J. A. (2009). Human induced pluripotent stem cells free of vector and transgene sequences. Science 324, 797-801.

1. A method of distinguishing a pluripotent stem cell (PSC) from a spontaneously differentiated cell (SDC), comprising identifying in said cell the presence of an alternatively spliced transcript which is preferentially expressed in said PSC compared to said SDC.

2. The method of claim 1, wherein said alternatively spliced transcript is unique to the PSC.

3. The method of claim 1, wherein said alternatively spliced transcript is expressed at a higher level in the PSC compared to the SDC.

4. The method of claim 1, wherein said alternatively spliced transcript is an exon-included transcript.

5. The method of claim 1, wherein said alternatively spliced transcript is an exon-excluded transcript.

6. The method of claim 1, wherein said alternatively spliced transcript is expressed from a nucleotide binding protein 2 (NUB2), a nucleoside diphosphate kinase A (NDKA), a purinergic receptor P2X, ligand-gated ion channel 5 (P2RX5), or a DNA cytosine-5-methyltransferase 3 beta (DNMT3B) gene.

7. The method of claim 6, wherein said alternatively spliced transcript is expressed from the DNMT3B gene.

8. The method of claim 7, wherein said alternatively spliced transcript comprises exon 10 of the DNMT3B gene.

9. The method of claim 8, wherein said alternatively spliced transcript comprises the nucleotide sequence:
(SEQ ID NO: 1)
AAGUCGAAGGUGCGUCGUGCAGGCAGUAGGAAAUUAGAAUCAAGG.

10. The method of claim 1, wherein said identifying is performed using a nucleic acid that binds said alternatively spliced transcript.

11. The method of claim 1, wherein said identifying is performed using primers that amplify said alternatively spliced transcript.

12. The method of claim 11, wherein said amplifying is performed by reverse transcription polymerase chain reaction (RT-PCR).

13. The method of claim 11, wherein said amplifying is performed by real time PCR.

14. The method of claim 1, wherein the alternatively spliced transcript is identified using a reporter gene construct.

15. The method of claim 14, wherein the reporter gene construct comprises: a promoter; a start codon; DNMT3Be10 sequence containing splice sites and intronic and exonic sequences; and a reporter gene.

16. The method of claim 15, wherein the promoter is a CMV promoter.

17. The method of claim 15, wherein the DNMT3Be10 sequence includes the 5′ splice site of intron 9/10; intron 9/10; the 3′ splice site between intron 9/10 and exon 10; the 5′ splice site between exon 10 and intron 10/11; the 3′ splice site between intron 10/11 and exon 11; and exon 11.

18. The method of claim 15 wherein the reporter gene is Green Fluorescent Protein (GFP).

19. A method of distinguishing a pluripotent stem cell (PSC) from a spontaneously differentiated cell (SDC), comprising identifying in said cell the presence of a polypeptide encoded by an alternatively spliced transcript which is preferentially expressed in said PSC compared to said SDC.

20. The method of claim 19, wherein said polypeptide is unique to the PSC.

21. The method of claim 19, wherein said polypeptide is expressed at a higher level in the PSC compared to the SDC.

22. The method of claim 19, wherein said polypeptide is encoded by a nucleotide binding protein 2 (NUB2), a nucleoside diphosphate kinase A (NDKA), a purinergic receptor P2X, ligand-gated ion channel 5 (P2RX5), or a DNA cytosine-5-methyltransferase 3 beta (DNMT3B) gene.

23. The method of claim 22, wherein said polypeptide is encoded by the DNMT3B gene.

24. The method of claim 23, wherein said polypeptide is encoded by exon 10 of the DNMT3B gene.

25. The method of claim 24, wherein said polypeptide comprises the sequence: KSKVRRAGSRKLESR (SEQ ID NO: 2).

26. The method of claim 19, wherein said identifying is performed using an antibody which binds the polypeptide.

27. The method of claim 26, wherein said antibody is a polyclonal antibody.

28. The method of claim 26, wherein said antibody is a monoclonal antibody.

29. An antibody to a polypeptide encoded by DNMT3B exon 10.

30. The antibody of claim 29, wherein said antibody binds the polypeptide sequence: KSKVRRAGSRKLESR (SEQ ID NO: 2).

31. The antibody of claim 29, wherein said antibody is a polyclonal antibody.

32. The antibody of claim 29, wherein said antibody is a monoclonal antibody.

33. The antibody of claim 31, wherein said antibody is SG1.

34. The antibody of claim 29, wherein said antibody is detectably labeled.


SEQUENCE LISTING (62 sequences)

SEQ ID NO: 1 - RNA, 45 nt, Homo sapiens
aagucgaaggugcgucgugcaggcaguaggaaauuagaaucaagg

SEQ ID NO: 2 - protein, 15 aa, Homo sapiens
KSKVRRAGSRKLESR

SEQ ID NO: 3 - DNA, 22 bp, Homo sapiens
cctggccggcgtcaggcacatc

SEQ ID NO: 4 - DNA, 22 bp, Homo sapiens
ggccatgtgctcatcggaggtc

SEQ ID NO: 5 - DNA, 22 bp, Homo sapiens
ccctgcagagtgctgtcatcac

SEQ ID NO: 6 - DNA, 22 bp, Homo sapiens
cgtttccagctgtaaccgcttc

SEQ ID NO: 7 - DNA, 23 bp, Homo sapiens
ttcacagcccaaatggcgggacc

SEQ ID NO: 8 - DNA, 22 bp, Homo sapiens
gcatgaacgcctgttcgccttc

SEQ ID NO: 9 - DNA, 24 bp, Homo sapiens
gcttcgtctactggagggcatgag

SEQ ID NO: 10 - DNA, 24 bp, Homo sapiens
ggcctcctggtacttgcgcttggc

SEQ ID NO: 11 - DNA, 25 bp, Homo sapiens
ccctgttagccgcccgctcagctct

SEQ ID NO: 12 - DNA, 24 bp, Homo sapiens
ctctctgattcacagggtctttgg

SEQ ID NO: 13 - DNA, 23 bp, Homo sapiens
cagaagctgttgggatgtagtcc

SEQ ID NO: 14 - DNA, 23 bp, Homo sapiens
agacaaagtggctgtcacaggtg

SEQ ID NO: 15 - DNA, 23 bp, Homo sapiens
gagtgcctgaaggagtataagtt

SEQ ID NO: 16 - DNA, 23 bp, Homo sapiens
cactgaaggggcctggccttggt

SEQ ID NO: 17 - DNA, 24 bp, Homo sapiens
ccactttggtgtttcataatctcc

SEQ ID NO: 18 - DNA, 24 bp, Homo sapiens
ccggacatcctgaaggtgctgctc

SEQ ID NO: 19 - DNA, 25 bp, Homo sapiens
gagtacctgaaccctctcgtggacg

SEQ ID NO: 20 - DNA, 27 bp, Homo sapiens
ccttgactctggctgtaatagccttcg

SEQ ID NO: 21 - DNA, 22 bp, Homo sapiens
cgtgcaagtgctgcgaaccacg

SEQ ID NO: 22 - DNA, 23 bp, Homo sapiens
cgcttgataatctctcccacaag

SEQ ID NO: 23 - DNA, 28 bp, Homo sapiens
gcaccggtgccactttaacggctgcagg

SEQ ID NO: 24 - DNA, 28 bp, Homo sapiens
ctccttccacggccggctctcagcctgg

SEQ ID NO: 25 - DNA, 24 bp, Homo sapiens
gctagggtgcgagctggcaagacc

SEQ ID NO: 26 - DNA, 28 bp, Homo sapiens
gtctttgccgttgttatagcaatttgtc

SEQ ID NO: 27 - DNA, 27 bp, Homo sapiens
ggccgttcttctggatgtttgagaatg

SEQ ID NO: 28 - DNA, 27 bp, Homo sapiens
gtcggatgacaggcacgctccaggacc

SEQ ID NO: 29 - DNA, 23 bp, Homo sapiens
cgggcgccggcggctttggtgac

SEQ ID NO: 30 - DNA, 24 bp, Homo sapiens
ggtcaccatggtaggcacggcgac

SEQ ID NO: 31 - DNA, 24 bp, Homo sapiens
gctagggtgcgagctggcaagacc

SEQ ID NO: 32 - DNA, 28 bp, Homo sapiens
taatttcctactgcctgcacgacgcacc

SEQ ID NO: 33 - DNA, 19 bp, Homo sapiens
cagtgcccgaaacccacac

SEQ ID NO: 34 - DNA, 20 bp, Homo sapiens
ggagacccagcagcctcaaa

SEQ ID NO: 35 - DNA, 21 bp, Homo sapiens
ggcactttcgatggtagtcgc

SEQ ID NO: 36 - DNA, 21 bp, Homo sapiens
cgtttctcaggctccctctcc

SEQ ID NO: 37 - DNA, 20 bp, Homo sapiens
aggcaagaaggtgggaatcc

SEQ ID NO: 38 - DNA, 20 bp, Homo sapiens
aggcaagaagcgctgataaa

SEQ ID NO: 39 - DNA, 40 bp, Homo sapiens
tccagcccagggagagaacgctgtgctgagaatgaaggca

SEQ ID NO: 40 - DNA, 20 bp, Homo sapiens
tccagcccagaatgaaggca

SEQ ID NO: 41 - DNA, 40 bp, Homo sapiens
catcagtcagtcctgggctggctcaccaagacccacagcc

SEQ ID NO: 42 - DNA, 20 bp, Homo sapiens
catcagtcagacccacagcc

SEQ ID NO: 43 - DNA, 40 bp, Homo sapiens
caaggaaaatgaagcctttgagctcggatgctttcctcaa

SEQ ID NO: 44 - DNA, 20 bp, Homo sapiens
caaggaaaatctttcctcaa

SEQ ID NO: 45 - DNA, 40 bp, Homo sapiens
cccccccacgaaattccttgtccctgtgaggtctatcatc

SEQ ID NO: 46 - DNA, 20 bp, Homo sapiens
cccccccacggtctatcatc

SEQ ID NO: 47 - DNA, 40 bp, Homo sapiens
gtttcttcagagcaggtatctgcggcccagcaagggggcc

SEQ ID NO: 48 - DNA, 20 bp, Homo sapiens
gtttcttgagctcaggggcc

SEQ ID NO: 49 - DNA, 40 bp, Homo sapiens
ctgttcccaggggccgtggttgaagaatatggatatgatg

SEQ ID NO: 50 - DNA, 20 bp, Homo sapiens
ctgttcccagggatatgatg

SEQ ID NO: 51 - DNA, 40 bp, Homo sapiens
agctggaaggaaccatggccgtgatacaggaaccatggcc

SEQ ID NO: 52 - DNA, 20 bp, Homo sapiens
agctggaaggaaccatggcc

SEQ ID NO: 53 - DNA, 40 bp, Homo sapiens
acgcacacaggagaaaagccactgtgacaggtgtttttcc

SEQ ID NO: 54 - DNA, 20 bp, Homo sapiens
acgcacacaggtgtttttcc

SEQ ID NO: 55 - DNA, 40 bp, Homo sapiens
acgcaaccagtggttaataaaggaaatacgagaacaagac

SEQ ID NO: 56 - DNA, 20 bp, Homo sapiens
acgcaaccagagaacaagac

SEQ ID NO: 57 - DNA, 60 bp, Homo sapiens
ggatgaacaggcccgtgatagatagccaagttaaagaaagagctcgaaaggatctttggc

SEQ ID NO: 58 - DNA, 40 bp, Homo sapiens
gttcctggagtgtaatccagggatgaacaggatctttggc

SEQ ID NO: 59 - DNA, 60 bp, Homo sapiens
tggttaataagtcgaaggtgcgtcgtgcaggcagtaggaaattagaatcaaggaaatacg

SEQ ID NO: 60 - DNA, 56 bp, Homo sapiens
agtccatacatttaatgtaatgttttttctgttttgttttgttttcccctcaaaag

SEQ ID NO: 61 - DNA, 57 bp, Homo sapiens
cattctctctggaccctcctaccaagcccacggctgcagtctaattacctttcacag

SEQ ID NO: 62 - DNA, 71 bp, Homo sapiens
acggtatttccttcctgtcttttgactgtgccctgttttctatgcactttctttctgatttctttgcatat